SEQUENCE LISTING (365 sequences)

SEQ ID NO: 1 - RNA, 22 nt, Homo sapiens
  hsa-miR-30a*
uguaaacauccucgacuggaag

SEQ ID NO: 2 - RNA, 22 nt, Homo sapiens
  hsa-miR-206
uggaauguaaggaagugugugg

SEQ ID NO: 3 - RNA, 18 nt, Homo sapiens
  hsa-miR-513_v9.1
uucacagggaggugucau

SEQ ID NO: 4 - RNA, 21 nt, Homo sapiens
  hsa-miR-583
caaagaggaaggucccauuac

SEQ ID NO: 5 - RNA, 22 nt, Homo sapiens
  hsa-miR-629*
guucucccaacguaagcccagc

SEQ ID NO: 6 - RNA, 22 nt, Homo sapiens
  hsa-miR-515-3p_v9.1
gagugccuucuuuuggagcguu

SEQ ID NO: 7 - RNA, 21 nt, Homo sapiens
  hsa-miR-571
ugaguuggccaucugagugag

SEQ ID NO: 8 - RNA, 20 nt, Homo sapiens
  hsa-miR-33b_v9.1
gugcauugcuguugcauugc

SEQ ID NO: 9 - RNA, 22 nt, Homo sapiens
  hsa-miR-125b
ucccugagacccuaacuuguga

SEQ ID NO: 10 - RNA, 22 nt, Homo sapiens
  hsa-miR-522_v9.1
aaaaugguucccuuuagagugu

SEQ ID NO: 11 - RNA, 23 nt, Homo sapiens
  hsa-miR-199b-5p
cccaguguuuagacuaucuguuc

SEQ ID NO: 12 - RNA, 23 nt, Homo sapiens
  hsa-miR-191_v9.1
caacggaaucccaaaagcagcug

SEQ ID NO: 13 - RNA, 23 nt, Homo sapiens
  hsa-miR-9
ucuuugguuaucuagcuguauga

SEQ ID NO: 14 - RNA, 22 nt, Homo sapiens
  hsa-miR-1_v9.1
uggaauguaaagaaguauguau

SEQ ID NO: 15 - RNA, 22 nt, Homo sapiens
  hsa-miR-660
uacccauugcauaucggaguug

SEQ ID NO: 16 - RNA, 21 nt, Homo sapiens
  hsa-miR-765
uggaggagaaggaaggugaug

SEQ ID NO: 17 - RNA, 22 nt, Homo sapiens
  hsa-miR-146b-5p
ugagaacugaauuccauaggcu

SEQ ID NO: 18 - RNA, 23 nt, Homo sapiens
  hsa-miR-135b_v9.1
uauggcuuuucauuccuauguga

SEQ ID NO: 19 - RNA, 23 nt, Homo sapiens
  hsa-miR-523_v9.1
gaacgcgcuucccuauagagggu

SEQ ID NO: 20 - RNA, 22 nt, Homo sapiens
  hsa-miR-381
uauacaagggcaagcucucugu

SEQ ID NO: 21 - RNA, 22 nt, Homo sapiens
  hsa-miR-490-3p
caaccuggaggacuccaugcug

SEQ ID NO: 22 - RNA, 22 nt, Homo sapiens
  hsa-miR-210
cugugcgugugacagcggcuga

SEQ ID NO: 23 - RNA, 22 nt, Homo sapiens
  hsa-miR-200a
uaacacugucugguaacgaugu

SEQ ID NO: 24 - RNA, 21 nt, Homo sapiens
  hsa-miR-520b
aaagugcuuccuuuuagaggg

SEQ ID NO: 25 - RNA, 20 nt, Homo sapiens
  hsa-miR-147
guguguggaaaugcuucugc

SEQ ID NO: 26 - RNA, 22 nt, Homo sapiens
  hsa-miR-16
uagcagcacguaaauauuggcg

SEQ ID NO: 27 - RNA, 20 nt, Homo sapiens
  hsa-miR-591
agaccauggguucucauugu

SEQ ID NO: 28 - RNA, 23 nt, Homo sapiens
  hsa-miR-668
ugucacucggcucggcccacuac

SEQ ID NO: 29 - RNA, 21 nt, Homo sapiens
  hsa-miR-101_v9.1
uacaguacugugauaacugaa

SEQ ID NO: 30 - RNA, 22 nt, Homo sapiens
  hsa-miR-519b_v9.1
aaagugcauccuuuuagagguu

SEQ ID NO: 31 - RNA, 21 nt, Homo sapiens
  hsa-miR-128b_v9.1
ucacagugaaccggucucuuu

SEQ ID NO: 32 - RNA, 21 nt, Homo sapiens
  hsa-miR-650
aggaggcagcgcucucaggac

SEQ ID NO: 33 - RNA, 21 nt, Homo sapiens
  hsa-miR-518d-3p
caaagcgcuucccuuuggagc

SEQ ID NO: 34 - RNA, 23 nt, Homo sapiens
  hsa-miR-423_v9.1
agcucggucugaggccccucagu

SEQ ID NO: 35 - RNA, 23 nt, Homo sapiens
  hsa-miR-19b
ugugcaaauccaugcaaaacuga

SEQ ID NO: 36 - RNA, 22 nt, Homo sapiens
  hsa-miR-586
uaugcauuguauuuuuaggucc

SEQ ID NO: 37 - RNA, 23 nt, Homo sapiens
  hsa-miR-639
aucgcugcgguugcgagcgcugu

SEQ ID NO: 38 - RNA, 22 nt, Homo sapiens
  hsa-miR-196a_v9.1
uagguaguuucauguuguuggg

SEQ ID NO: 39 - RNA, 22 nt, Homo sapiens
  hsa-miR-187_v9.1
ucgugucuuguguugcagccgg

SEQ ID NO: 40 - RNA, 22 nt, Homo sapiens
  hsa-miR-520f_v9.1
aagugcuuccuuuuagaggguu

SEQ ID NO: 41 - RNA, 20 nt, Homo sapiens
  hsa-miR-144_v9.1
uacaguauagaugauguacu

SEQ ID NO: 42 - RNA, 23 nt, Homo sapiens
  hsa-miR-767-5p
ugcaccaugguugucugagcaug

SEQ ID NO: 43 - RNA, 22 nt, Homo sapiens
  hsa-miR-592
uugugucaauaugcgaugaugu

SEQ ID NO: 44 - RNA, 23 nt, Homo sapiens
  hsa-miR-492
aggaccugcgggacaagauucuu

SEQ ID NO: 45 - RNA, 22 nt, Homo sapiens
  hsa-miR-95
uucaacggguauuuauugagca

SEQ ID NO: 46 - RNA, 21 nt, Homo sapiens
  hsa-miR-422b_v9.1
acuggacuuggagucagaagg

SEQ ID NO: 47 - RNA, 22 nt, Homo sapiens
  hsa-miR-526a_v9.1
cucuagagggaagcacuuucug

SEQ ID NO: 48 - RNA, 23 nt, Homo sapiens
  hsa-miR-34b*
uaggcagugucauuagcugauug

SEQ ID NO: 49 - RNA, 23 nt, Homo sapiens
  hsa-miR-302b
uaagugcuuccauguuuuaguag

SEQ ID NO: 50 - RNA, 23 nt, Homo sapiens
  hsa-miR-602
gacacgggcgacagcugcggccc

SEQ ID NO: 51 - RNA, 22 nt, Homo sapiens
  hsa-miR-15b
uagcagcacaucaugguuuaca

SEQ ID NO: 52 - RNA, 23 nt, Homo sapiens
  hsa-miR-30c
uguaaacauccuacacucucagc

SEQ ID NO: 53 - RNA, 19 nt, Homo sapiens
  hsa-miR-585
ugggcguaucuguaugcua

SEQ ID NO: 54 - RNA, 22 nt, Homo sapiens
  hsa-miR-493-3p_v9.1
ugaaggucuacugugugccagg

SEQ ID NO: 55 - RNA, 22 nt, Homo sapiens
  hsa-miR-518a-3p
gaaagcgcuucccuuugcugga

SEQ ID NO: 56 - RNA, 22 nt, Homo sapiens
  hsa-miR-550*
ugucuuacucccucaggcacau

SEQ ID NO: 57 - RNA, 22 nt, Homo sapiens
  hsa-miR-483_v9.1
aagacgggaggaaagaagggag

SEQ ID NO: 58 - RNA, 23 nt, Homo sapiens
  hsa-miR-17-5p_v9.1
caaagugcuuacagugcagguag

SEQ ID NO: 59 - RNA, 22 nt, Homo sapiens
  hsa-miR-500*
augcaccugggcaaggauucug

SEQ ID NO: 60 - RNA, 21 nt, Homo sapiens
  hsa-miR-520e
aaagugcuuccuuuuugaggg

SEQ ID NO: 61 - RNA, 24 nt, Homo sapiens
  hsa-miR-520g
acaaagugcuucccuuuagagugu

SEQ ID NO: 62 - RNA, 22 nt, Homo sapiens
  hsa-miR-615_v9.1
uccgagccugggucucccucuu

SEQ ID NO: 63 - RNA, 21 nt, Homo sapiens
  hsa-miR-26b_v9.1
uucaaguaauucaggauaggu

SEQ ID NO: 64 - RNA, 22 nt, Homo sapiens
  hsa-miR-449a
uggcaguguauuguuagcuggu

SEQ ID NO: 65 - RNA, 21 nt, Homo sapiens
  hsa-miR-499_v9.1
uuaagacuugcagugauguuu

SEQ ID NO: 66 - RNA, 22 nt, Homo sapiens
  hsa-miR-196b_v9.1
uagguaguuuccuguuguuggg

SEQ ID NO: 67 - RNA, 22 nt, Homo sapiens
  hsa-miR-203
gugaaauguuuaggaccacuag

SEQ ID NO: 68 - RNA, 22 nt, Homo sapiens
  hsa-miR-548d-3p
caaaaaccacaguuucuuuugc

SEQ ID NO: 69 - RNA, 22 nt, Homo sapiens
  hsa-miR-548b-3p
caagaaccucaguugcuuuugu

SEQ ID NO: 70 - RNA, 22 nt, Homo sapiens
  hsa-miR-126_v9.1
ucguaccgugaguaauaaugcg

SEQ ID NO: 71 - RNA, 23 nt, Homo sapiens
  hsa-miR-518c_v9.1
caaagcgcuucucuuuagagugu

SEQ ID NO: 72 - RNA, 21 nt, Homo sapiens
  hsa-miR-337_v9.1
gaacggcuucauacaggaguu

SEQ ID NO: 73 - RNA, 23 nt, Homo sapiens
  hsa-miR-605
uaaaucccauggugccuucuccu

SEQ ID NO: 74 - RNA, 21 nt, Homo sapiens
  hsa-miR-551b
gcgacccauacuugguuucag

SEQ ID NO: 75 - RNA, 22 nt, Homo sapiens
  hsa-miR-92_v9.1
uauugcacuugucccggccugu

SEQ ID NO: 76 - RNA, 21 nt, Homo sapiens
  hsa-miR-224_v9.1
caagucacuagugguuccguu

SEQ ID NO: 77 - RNA, 19 nt, Homo sapiens
  hsa-miR-645
ucuaggcugguacugcuga

SEQ ID NO: 78 - RNA, 22 nt, Homo sapiens
  hsa-miR-190
ugauauguuugauauauuaggu

SEQ ID NO: 79 - RNA, 23 nt, Homo sapiens
  hsa-miR-106a_v9.1
aaaagugcuuacagugcagguag

SEQ ID NO: 80 - RNA, 22 nt, Homo sapiens
  hsa-miR-517b
ucgugcaucccuuuagaguguu

SEQ ID NO: 81 - RNA, 22 nt, Homo sapiens
  hsa-miR-185_v9.1
uggagagaaaggcaguuccuga

SEQ ID NO: 82 - RNA, 22 nt, Homo sapiens
  hsa-let-7a
ugagguaguagguuguauaguu

SEQ ID NO: 83 - RNA, 21 nt, Homo sapiens
  hsa-miR-129-5p
cuuuuugcggucugggcuugc

SEQ ID NO: 84 - RNA, 22 nt, Homo sapiens
  hsa-miR-299-3p
uaugugggaugguaaaccgcuu

SEQ ID NO: 85 - RNA, 22 nt, Homo sapiens
  hsa-miR-25
cauugcacuugucucggucuga

SEQ ID NO: 86 - RNA, 19 nt, Homo sapiens
  hsa-miR-604
aggcugcggaauucaggac

SEQ ID NO: 87 - RNA, 23 nt, Homo sapiens
  hsa-miR-498
uuucaagccagggggcguuuuuc

SEQ ID NO: 88 - RNA, 22 nt, Homo sapiens
  hsa-miR-449b
aggcaguguauuguuagcuggc

SEQ ID NO: 89 - RNA, 23 nt, Homo sapiens
  hsa-miR-302a*_v9.1
acuuaaacguggauguacuugcu

SEQ ID NO: 90 - RNA, 20 nt, Homo sapiens
  hsa-miR-613
aggaauguuccuucuuugcc

SEQ ID NO: 91 - RNA, 22 nt, Homo sapiens
  hsa-miR-519d_v9.1
caaagugccucccuuuagagug

SEQ ID NO: 92 - RNA, 23 nt, Homo sapiens
  hsa-miR-425
aaugacacgaucacucccguuga

SEQ ID NO: 93 - RNA, 22 nt, Homo sapiens
  hsa-miR-328
cuggcccucucugcccuuccgu

SEQ ID NO: 94 - RNA, 24 nt, Homo sapiens
  hsa-miR-619
gaccuggacauguuugugcccagu

SEQ ID NO: 95 - RNA, 22 nt, Homo sapiens
  hsa-miR-380
uauguaauaugguccacaucuu

SEQ ID NO: 96 - RNA, 21 nt, Homo sapiens
  hsa-miR-507
uuuugcaccuuuuggagugaa

SEQ ID NO: 97 - RNA, 23 nt, Homo sapiens
  hsa-miR-302d
uaagugcuuccauguuugagugu

SEQ ID NO: 98 - RNA, 22 nt, Homo sapiens
  hsa-miR-630
aguauucuguaccagggaaggu

SEQ ID NO: 99 - RNA, 22 nt, Homo sapiens
  hsa-miR-433
aucaugaugggcuccucggugu

SEQ ID NO: 100 - RNA, 22 nt, Homo sapiens
  hsa-miR-493*
uuguacaugguaggcuuucauu

SEQ ID NO: 101 - RNA, 22 nt, Homo sapiens
  hsa-let-7f
ugagguaguagauuguauaguu

SEQ ID NO: 102 - RNA, 22 nt, Homo sapiens
  hsa-miR-184
uggacggagaacugauaagggu

SEQ ID NO: 103 - RNA, 22 nt, Homo sapiens
  hsa-miR-422a_v9.1
acuggacuuagggucagaaggc

SEQ ID NO: 104 - RNA, 21 nt, Homo sapiens
  hsa-miR-587
uuuccauaggugaugagucac

SEQ ID NO: 105 - RNA, 21 nt, Homo sapiens
  hsa-miR-296-5p
agggcccccccucaauccugu

SEQ ID NO: 106 - RNA, 24 nt, Homo sapiens
  hsa-miR-573
cugaagugauguguaacugaucag

SEQ ID NO: 107 - RNA, 21 nt, Homo sapiens
  hsa-miR-192
cugaccuaugaauugacagcc

SEQ ID NO: 108 - RNA, 22 nt, Homo sapiens
  hsa-miR-556_v9.1
gaugagcucauuguaauaugag

SEQ ID NO: 109 - RNA, 22 nt, Homo sapiens
  hsa-miR-374a
uuauaauacaaccugauaagug

SEQ ID NO: 110 - RNA, 23 nt, Homo sapiens
  hsa-miR-107
agcagcauuguacagggcuauca

SEQ ID NO: 111 - RNA, 22 nt, Homo sapiens
  hsa-miR-659
cuugguucagggagggucccca

SEQ ID NO: 112 - RNA, 21 nt, Homo sapiens
  hsa-miR-628_v9.1
ucuaguaagaguggcagucga

SEQ ID NO: 113 - RNA, 20 nt, Homo sapiens
  hsa-miR-609
aggguguuucucucaucucu

SEQ ID NO: 114 - RNA, 23 nt, Homo sapiens
  hsa-miR-18a*_v9.1
acugcccuaagugcuccuucugg

SEQ ID NO: 115 - RNA, 22 nt, Homo sapiens
  hsa-miR-485-3p
gucauacacggcucuccucucu

SEQ ID NO: 116 - RNA, 22 nt, Homo sapiens
  hsa-miR-141
uaacacugucugguaaagaugg

SEQ ID NO: 117 - RNA, 22 nt, Homo sapiens
  hsa-miR-519c-3p
aaagugcaucuuuuuagaggau

SEQ ID NO: 118 - RNA, 22 nt, Homo sapiens
  hsa-miR-132
uaacagucuacagccauggucg

SEQ ID NO: 119 - RNA, 22 nt, Homo sapiens
  hsa-miR-624*
uaguaccaguaccuuguguuca

SEQ ID NO: 120 - RNA, 23 nt, Homo sapiens
  hsa-miR-20a
uaaagugcuuauagugcagguag

SEQ ID NO: 121 - RNA, 23 nt, Homo sapiens
  hsa-miR-421
aucaacagacauuaauugggcgc

SEQ ID NO: 122 - RNA, 22 nt, Homo sapiens
  hsa-let-7e_v9.1
ugagguaggagguuguauaguu

SEQ ID NO: 123 - RNA, 21 nt, Homo sapiens
  hsa-miR-212
uaacagucuccagucacggcc

SEQ ID NO: 124 - RNA, 23 nt, Homo sapiens
  hsa-miR-20b
caaagugcucauagugcagguag

SEQ ID NO: 125 - RNA, 22 nt, Homo sapiens
  hsa-miR-576_v9.1
auucuaauuucuccacgucuuu

SEQ ID NO: 126 - RNA, 23 nt, Homo sapiens
  hsa-miR-526b_v9.1
cucuugagggaagcacuuucugu

SEQ ID NO: 127 - RNA, 22 nt, Homo sapiens
  hsa-miR-649
aaaccuguguuguucaagaguc

SEQ ID NO: 128 - RNA, 22 nt, Homo sapiens
  hsa-miR-519a_v9.1
aaagugcauccuuuuagagugu

SEQ ID NO: 129 - RNA, 22 nt, Homo sapiens
  hsa-miR-100
aacccguagauccgaacuugug

SEQ ID NO: 130 - RNA, 21 nt, Homo sapiens
  hsa-miR-607
guucaaauccagaucuauaac

SEQ ID NO: 131 - RNA, 22 nt, Homo sapiens
  hsa-miR-643
acuuguaugcuagcucagguag

SEQ ID NO: 132 - RNA, 22 nt, Homo sapiens
  hsa-let-7g_v9.1
ugagguaguaguuuguacaguu

SEQ ID NO: 133 - RNA, 22 nt, Homo sapiens
  hsa-miR-98
ugagguaguaaguuguauuguu

SEQ ID NO: 134 - RNA, 21 nt, Homo sapiens
  hsa-miR-195
uagcagcacagaaauauuggc

SEQ ID NO: 135 - RNA, 22 nt, Homo sapiens
  hsa-miR-603
cacacacugcaauuacuuuugc

SEQ ID NO: 136 - RNA, 22 nt, Homo sapiens
  hsa-miR-448
uugcauauguaggaugucccau

SEQ ID NO: 137 - RNA, 22 nt, Homo sapiens
  hsa-miR-34a_v9.1
uggcagugucuuagcugguugu

SEQ ID NO: 138 - RNA, 22 nt, Homo sapiens
  hsa-miR-539
ggagaaauuauccuuggugugu

SEQ ID NO: 139 - RNA, 22 nt, Homo sapiens
  hsa-miR-520d*_v9.1
aaagugcuucucuuuggugggu

SEQ ID NO: 140 - RNA, 20 nt, Homo sapiens
  hsa-miR-568
auguauaaauguauacacac

SEQ ID NO: 141 - RNA, 22 nt, Homo sapiens
  hsa-miR-299-5p
ugguuuaccgucccacauacau

SEQ ID NO: 142 - RNA, 23 nt, Homo sapiens
  hsa-miR-34c-5p
aggcaguguaguuagcugauugc

SEQ ID NO: 143 - RNA, 21 nt, Homo sapiens
  hsa-miR-152_v9.1
ucagugcaugacagaacuugg

SEQ ID NO: 144 - RNA, 21 nt, Homo sapiens
  hsa-miR-553
aaaacggugagauuuuguuuu

SEQ ID NO: 145 - RNA, 22 nt, Homo sapiens
  hsa-miR-509_v9.1
ugauugguacgucuguggguag

SEQ ID NO: 146 - RNA, 18 nt, Homo sapiens
  hsa-miR-516a-3p
ugcuuccuuucagagggu

SEQ ID NO: 147 - RNA, 22 nt, Homo sapiens
  hsa-miR-122a_v9.1
uggagugugacaaugguguuug

SEQ ID NO: 148 - RNA, 19 nt, Homo sapiens
  hsa-miR-644
aguguggcuuucuuagagc

SEQ ID NO: 149 - RNA, 20 nt, Homo sapiens
  hsa-miR-599
guugugucaguuuaucaaac

SEQ ID NO: 150 - RNA, 21 nt, Homo sapiens
  hsa-miR-514_v9.1
auugacacuucugugaguaga

SEQ ID NO: 151 - RNA, 22 nt, Homo sapiens
  hsa-miR-512-3p
aagugcugucauagcugagguc

SEQ ID NO: 152 - RNA, 21 nt, Homo sapiens
  hsa-miR-578
cuucuugugcucuaggauugu

SEQ ID NO: 153 - RNA, 22 nt, Homo sapiens
  hsa-miR-561
caaaguuuaagauccuugaagu

SEQ ID NO: 154 - RNA, 23 nt, Homo sapiens
  hsa-miR-149_v9.1
ucuggcuccgugucuucacuccc

SEQ ID NO: 155 - RNA, 21 nt, Homo sapiens
  hsa-miR-625_v9.1
agggggaaaguucuauagucc

SEQ ID NO: 156 - RNA, 22 nt, Homo sapiens
  hsa-miR-134_v9.1
ugugacugguugaccagagggg

SEQ ID NO: 157 - RNA, 23 nt, Homo sapiens
  hsa-miR-454-3p_v9.1
uagugcaauauugcuuauagggu

SEQ ID NO: 158 - RNA, 22 nt, Homo sapiens
  hsa-miR-373*
acucaaaaugggggcgcuuucc

SEQ ID NO: 159 - RNA, 22 nt, Homo sapiens
  hsa-miR-198_v9.1
gguccagaggggagauagguuc

SEQ ID NO: 160 - RNA, 21 nt, Homo sapiens
  hsa-miR-610
ugagcuaaaugugugcuggga

SEQ ID NO: 161 - RNA, 25 nt, Homo sapiens
  hsa-miR-658
ggcggagggaaguagguccguuggu

SEQ ID NO: 162 - RNA, 22 nt, Homo sapiens
  hsa-miR-383
agaucagaaggugauuguggcu

SEQ ID NO: 163 - RNA, 22 nt, Homo sapiens
  hsa-miR-30b
uguaaacauccuacacucagcu

SEQ ID NO: 164 - RNA, 22 nt, Homo sapiens
  hsa-miR-663
aggcggggcgccgcgggaccgc

SEQ ID NO: 165 - RNA, 21 nt, Homo sapiens
  hsa-miR-552
aacaggugacugguuagacaa

SEQ ID NO: 166 - RNA, 22 nt, Homo sapiens
  hsa-miR-519e_v9.1
aagugccuccuuuuagaguguu

SEQ ID NO: 167 - RNA, 23 nt, Homo sapiens
  hsa-miR-453_v9.1
agguuguccguggugaguucgca

SEQ ID NO: 168 - RNA, 23 nt, Homo sapiens
  hsa-miR-29b
uagcaccauuugaaaucaguguu

SEQ ID NO: 169 - RNA, 22 nt, Homo sapiens
  hsa-miR-127-3p
ucggauccgucugagcuuggcu

SEQ ID NO: 170 - RNA, 22 nt, Homo sapiens
  hsa-miR-367
aauugcacuuuagcaaugguga

SEQ ID NO: 171 - RNA, 22 nt, Homo sapiens
  hsa-miR-545_v9.1
ucagcaaacauuuauugugugc

SEQ ID NO: 172 - RNA, 22 nt, Homo sapiens
  hsa-miR-532-5p
caugccuugaguguaggaccgu

SEQ ID NO: 173 - RNA, 22 nt, Homo sapiens
  hsa-miR-32_v9.1
uauugcacauuacuaaguugca

SEQ ID NO: 174 - RNA, 22 nt, Homo sapiens
  hsa-miR-516b
aucuggagguaagaagcacuuu

SEQ ID NO: 175 - RNA, 22 nt, Homo sapiens
  hsa-miR-200b_v9.1
uaauacugccugguaaugauga

SEQ ID NO: 176 - RNA, 21 nt, Homo sapiens
  hsa-miR-595
gaagugugccguggugugucu

SEQ ID NO: 177 - RNA, 24 nt, Homo sapiens
  hsa-miR-362-5p
aauccuuggaaccuaggugugagu

SEQ ID NO: 178 - RNA, 22 nt, Homo sapiens
  hsa-miR-99a
aacccguagauccgaucuugug

SEQ ID NO: 179 - RNA, 22 nt, Homo sapiens
  hsa-miR-601
uggucuaggauuguuggaggag

SEQ ID NO: 180 - RNA, 22 nt, Homo sapiens
  hsa-miR-191*
gcugcgcuuggauuucgucccc

SEQ ID NO: 181 - RNA, 21 nt, Homo sapiens
  hsa-miR-128a_v9.1
ucacagugaaccggucucuuu

SEQ ID NO: 182 - RNA, 22 nt, Homo sapiens
  hsa-miR-651
uuuaggauaagcuugacuuuug

SEQ ID NO: 183 - RNA, 22 nt, Homo sapiens
  hsa-miR-590-5p
gagcuuauucauaaaagugcag

SEQ ID NO: 184 - RNA, 22 nt, Homo sapiens
  hsa-miR-501-5p
aauccuuugucccugggugaga

SEQ ID NO: 185 - RNA, 22 nt, Homo sapiens
  hsa-miR-526b*
gaaagugcuuccuuuuagaggc

SEQ ID NO: 186 - RNA, 22 nt, Homo sapiens
  hsa-miR-487b
aaucguacagggucauccacuu

SEQ ID NO: 187 - RNA, 25 nt, Homo sapiens
  hsa-miR-608
aggggugguguugggacagcuccgu

SEQ ID NO: 188 - RNA, 23 nt, Homo sapiens
  hsa-miR-508-3p
ugauuguagccuuuuggaguaga

SEQ ID NO: 189 - RNA, 23 nt, Homo sapiens
  hsa-miR-19a
ugugcaaaucuaugcaaaacuga

SEQ ID NO: 190 - RNA, 22 nt, Homo sapiens
  hsa-let-7i_v9.1
ugagguaguaguuugugcuguu

SEQ ID NO: 191 - RNA, 22 nt, Homo sapiens
  hsa-miR-518f*
cucuagagggaagcacuuucuc

SEQ ID NO: 192 - RNA, 23 nt, Homo sapiens
  hsa-miR-181d_v9.1
aacauucauuguugucggugggu

SEQ ID NO: 193 - RNA, 22 nt, Homo sapiens
  hsa-miR-302c*
uuuaacauggggguaccugcug

SEQ ID NO: 194 - RNA, 23 nt, Homo sapiens
  hsa-miR-373
gaagugcuucgauuuuggggugu

SEQ ID NO: 195 - RNA, 22 nt, Homo sapiens
  hsa-miR-517c
aucgugcauccuuuuagagugu

SEQ ID NO: 196 - RNA, 22 nt, Homo sapiens
  hsa-let-7b
ugagguaguagguugugugguu

SEQ ID NO: 197 - RNA, 23 nt, Homo sapiens
  hsa-miR-769-3p
cugggaucuccggggucuugguu

SEQ ID NO: 198 - RNA, 22 nt, Homo sapiens
  hsa-miR-655
auaauacaugguuaaccucuuu

SEQ ID NO: 199 - RNA, 23 nt, Homo sapiens
  hsa-miR-432
ucuuggaguaggucauugggugg

SEQ ID NO: 200 - RNA, 23 nt, Homo sapiens
  hsa-miR-325
ccuaguagguguccaguaagugu

SEQ ID NO: 201 - RNA, 23 nt, Homo sapiens
  hsa-miR-135a
uauggcuuuuuauuccuauguga

SEQ ID NO: 202 - RNA, 23 nt, Homo sapiens
  hsa-miR-7_v9.1
uggaagacuagugauuuuguugu

SEQ ID NO: 203 - RNA, 22 nt, Homo sapiens
  hsa-miR-193a_v9.1
aacuggccuacaaagucccagu

SEQ ID NO: 204 - RNA, 21 nt, Homo sapiens
  hsa-miR-27b
uucacaguggcuaaguucugc

SEQ ID NO: 205 - RNA, 20 nt, Homo sapiens
  hsa-miR-202_v9.1
agagguauagggcaugggaa

SEQ ID NO: 206 - RNA, 22 nt, Homo sapiens
  hsa-let-7d_v9.1
agagguaguagguugcauaguu

SEQ ID NO: 207 - RNA, 20 nt, Homo sapiens
  hsa-miR-562
aaaguagcuguaccauuugc

SEQ ID NO: 208 - RNA, 22 nt, Homo sapiens
  hsa-miR-183_v9.1
uauggcacugguagaauucacu

SEQ ID NO: 209 - RNA, 21 nt, Homo sapiens
  hsa-miR-596
aagccugcccggcuccucggg

SEQ ID NO: 210 - RNA, 21 nt, Homo sapiens
  hsa-miR-622
acagucugcugagguuggagc

SEQ ID NO: 211 - RNA, 22 nt, Homo sapiens
  hsa-miR-370_v9.1
gccugcugggguggaaccuggu

SEQ ID NO: 212 - RNA, 23 nt, Homo sapiens
  hsa-miR-155_v9.1
uuaaugcuaaucgugauaggggu

SEQ ID NO: 213 - RNA, 23 nt, Homo sapiens
  hsa-miR-302c
uaagugcuuccauguuucagugg

SEQ ID NO: 214 - RNA, 21 nt, Homo sapiens
  hsa-miR-524-3p
gaaggcgcuucccuuuggagu

SEQ ID NO: 215 - RNA, 22 nt, Homo sapiens
  hsa-miR-363
aauugcacgguauccaucugua

SEQ ID NO: 216 - RNA, 23 nt, Homo sapiens
  hsa-miR-582-5p
uuacaguuguucaaccaguuacu

SEQ ID NO: 217 - RNA, 23 nt, Homo sapiens
  hsa-miR-301a
cagugcaauaguauugucaaagc

SEQ ID NO: 218 - RNA, 23 nt, Homo sapiens
  hsa-miR-138_v9.1
agcugguguugugaaucaggccg

SEQ ID NO: 219 - RNA, 20 nt, Homo sapiens
  hsa-miR-520d_v9.1
cuacaaagggaagcccuuuc

SEQ ID NO: 220 - RNA, 21 nt, Homo sapiens
  hsa-miR-518f_v9.1
gaaagcgcuucucuuuagagg

SEQ ID NO: 221 - RNA, 22 nt, Homo sapiens
  hsa-miR-28-5p
aaggagcucacagucuauugag

SEQ ID NO: 222 - RNA, 22 nt, Homo sapiens
  hsa-let-7c
ugagguaguagguuguaugguu

SEQ ID NO: 223 - RNA, 22 nt, Homo sapiens
  hsa-miR-369-5p
agaucgaccguguuauauucgc

SEQ ID NO: 224 - RNA, 22 nt, Homo sapiens
  hsa-miR-450_v9.1
uuuugcgauguguuccuaauau

SEQ ID NO: 225 - RNA, 23 nt, Homo sapiens
  hsa-miR-137
uuauugcuuaagaauacgcguag

SEQ ID NO: 226 - RNA, 22 nt, Homo sapiens
  hsa-miR-524-5p
cuacaaagggaagcacuuucuc

SEQ ID NO: 227 - RNA, 22 nt, Homo sapiens
  hsa-miR-520h
acaaagugcuucccuuuagagu

SEQ ID NO: 228 - RNA, 20 nt, Homo sapiens
  hsa-miR-572
guccgcucggcgguggccca

SEQ ID NO: 229 - RNA, 22 nt, Homo sapiens
  hsa-miR-495_v9.1
aaacaaacauggugcacuucuu

SEQ ID NO: 230 - RNA, 22 nt, Homo sapiens
  hsa-miR-758
uuugugaccugguccacuaacc

SEQ ID NO: 231 - RNA, 23 nt, Homo sapiens
  hsa-miR-10b_v9.1
uacccuguagaaccgaauuugug

SEQ ID NO: 232 - RNA, 22 nt, Homo sapiens
  hsa-miR-580
uugagaaugaugaaucauuagg

SEQ ID NO: 233 - RNA, 21 nt, Homo sapiens
  hsa-miR-215
augaccuaugaauugacagac

SEQ ID NO: 234 - RNA, 22 nt, Homo sapiens
  hsa-miR-378*
cuccugacuccagguccugugu

SEQ ID NO: 235 - RNA, 22 nt, Homo sapiens
  hsa-miR-30e*
cuuucagucggauguuuacagc

SEQ ID NO: 236 - RNA, 23 nt, Homo sapiens
  hsa-miR-93
caaagugcuguucgugcagguag

SEQ ID NO: 237 - RNA, 22 nt, Homo sapiens
  hsa-miR-521
aacgcacuucccuuuagagugu

SEQ ID NO: 238 - RNA, 21 nt, Homo sapiens
  hsa-miR-511
gugucuuuugcucugcaguca

SEQ ID NO: 239 - RNA, 21 nt, Homo sapiens
  hsa-miR-379_v9.1
ugguagacuauggaacguagg

SEQ ID NO: 240 - RNA, 21 nt, Homo sapiens
  hsa-miR-431
ugucuugcaggccgucaugca

SEQ ID NO: 241 - RNA, 22 nt, Homo sapiens
  hsa-miR-382
gaaguuguucgugguggauucg

SEQ ID NO: 242 - RNA, 19 nt, Homo sapiens
  hsa-miR-632
gugucugcuuccuguggga

SEQ ID NO: 243 - RNA, 21 nt, Homo sapiens
  hsa-miR-647
guggcugcacucacuuccuuc

SEQ ID NO: 244 - RNA, 24 nt, Homo sapiens
  hsa-miR-182_v9.1
uuuggcaaugguagaacucacacu

SEQ ID NO: 245 - RNA, 22 nt, Homo sapiens
  hsa-miR-154
uagguuauccguguugccuucg

SEQ ID NO: 246 - RNA, 21 nt, Homo sapiens
  hsa-miR-188_v9.1
caucccuugcaugguggaggg

SEQ ID NO: 247 - RNA, 22 nt, Homo sapiens
  hsa-miR-520a-3p
aaagugcuucccuuuggacugu

SEQ ID NO: 248 - RNA, 23 nt, Homo sapiens
  hsa-miR-302a
uaagugcuuccauguuuugguga

SEQ ID NO: 249 - RNA, 22 nt, Homo sapiens
  hsa-miR-451_v9.1
aaaccguuaccauuacugaguu

SEQ ID NO: 250 - RNA, 22 nt, Homo sapiens
  hsa-miR-133a_v9.1
uuugguccccuucaaccagcug

SEQ ID NO: 251 - RNA, 21 nt, Homo sapiens
  hsa-miR-126*
cauuauuacuuuugguacgcg

SEQ ID NO: 252 - RNA, 22 nt, Homo sapiens
  hsa-miR-24
uggcucaguucagcaggaacag

SEQ ID NO: 253 - RNA, 22 nt, Homo sapiens
  hsa-miR-30d
uguaaacauccccgacuggaag

SEQ ID NO: 254 - RNA, 23 nt, Homo sapiens
  hsa-miR-221
agcuacauugucugcuggguuuc

SEQ ID NO: 255 - RNA, 24 nt, Homo sapiens
  hsa-miR-125a_v9.1
ucccugagacccuuuaaccuguga

SEQ ID NO: 256 - RNA, 23 nt, Homo sapiens
  hsa-miR-335
ucaagagcaauaacgaaaaaugu

SEQ ID NO: 257 - RNA, 23 nt, Homo sapiens
  hsa-miR-342_v9.1
ucucacacagaaaucgcacccgu

SEQ ID NO: 258 - RNA, 23 nt, Homo sapiens
  hsa-miR-324-5p
cgcauccccuagggcauuggugu

SEQ ID NO: 259 - RNA, 23 nt, Homo sapiens
  hsa-miR-18a_v9.1
uaaggugcaucuagugcagauag

SEQ ID NO: 260 - RNA, 23 nt, Homo sapiens
  hsa-miR-181a
aacauucaacgcugucggugagu

SEQ ID NO: 261 - RNA, 19 nt, Homo sapiens
  hsa-miR-575
gagccaguuggacaggagc

SEQ ID NO: 262 - RNA, 22 nt, Homo sapiens
  hsa-miR-365
uaaugccccuaaaaauccuuau

SEQ ID NO: 263 - RNA, 21 nt, Homo sapiens
  hsa-miR-27a
uucacaguggcuaaguuccgc

SEQ ID NO: 264 - RNA, 22 nt, Homo sapiens
  hsa-miR-130b
cagugcaaugaugaaagggcau

SEQ ID NO: 265 - RNA, 22 nt, Homo sapiens
  hsa-miR-193b_v9.1
aacuggcccucaaagucccgcu

SEQ ID NO: 266 - RNA, 21 nt, Homo sapiens
  hsa-miR-222_v9.1
agcuacaucuggcuacugggu

SEQ ID NO: 267 - RNA, 23 nt, Homo sapiens
  hsa-miR-200c_v9.1
uaauacugccggguaaugaugga

SEQ ID NO: 268 - RNA, 21 nt, Homo sapiens
  hsa-miR-106b
uaaagugcugacagugcagau

SEQ ID NO: 269 - RNA, 22 nt, Homo sapiens
  hsa-miR-361-5p
uuaucagaaucuccagggguac

SEQ ID NO: 270 - RNA, 20 nt, Homo sapiens
  hsa-miR-326
ccucugggcccuuccuccag

SEQ ID NO: 271 - RNA, 22 nt, Homo sapiens
  hsa-miR-148b
ucagugcaucacagaacuuugu

SEQ ID NO: 272 - RNA, 21 nt, Homo sapiens
  hsa-miR-151-3p
cuagacugaagcuccuugagg

SEQ ID NO: 273 - RNA, 21 nt, Homo sapiens
  hsa-miR-410
aauauaacacagauggccugu

SEQ ID NO: 274 - RNA, 20 nt, Homo sapiens
  hsa-miR-527_v9.1
cugcaaagggaagcccuuuc

SEQ ID NO: 275 - RNA, 22 nt, Homo sapiens
  hsa-miR-205
uccuucauuccaccggagucug

SEQ ID NO: 276 - RNA, 22 nt, Homo sapiens
  hsa-miR-99b
cacccguagaaccgaccuugcg

SEQ ID NO: 277 - RNA, 22 nt, Homo sapiens
  hsa-miR-15a
uagcagcacauaaugguuugug

SEQ ID NO: 278 - RNA, 21 nt, Homo sapiens
  hsa-miR-23a
aucacauugccagggauuucc

SEQ ID NO: 279 - RNA, 23 nt, Homo sapiens
  hsa-miR-636_v9.1
ugugcuugcucgucccgcccgca

SEQ ID NO: 280 - RNA, 25 nt, Homo sapiens
  hsa-miR-638
agggaucgcgggcggguggcggccu

SEQ ID NO: 281 - RNA, 22 nt, Homo sapiens
  hsa-miR-345_v9.1
gcugacuccuaguccagggcuc

SEQ ID NO: 282 - RNA, 22 nt, Homo sapiens
  hsa-miR-519e*
uucuccaaaagggagcacuuuc

SEQ ID NO: 283 - RNA, 22 nt, Homo sapiens
  hsa-miR-654-5p
uggugggccgcagaacaugugc

SEQ ID NO: 284 - RNA, 23 nt, Homo sapiens
  hsa-miR-142-3p
uguaguguuuccuacuuuaugga

SEQ ID NO: 285 - RNA, 21 nt, Homo sapiens
  hsa-miR-31_v9.1
aggcaagaugcuggcauagcu

SEQ ID NO: 286 - RNA, 22 nt, Homo sapiens
  hsa-miR-133b
uuugguccccuucaaccagcua

SEQ ID NO: 287 - RNA, 22 nt, Homo sapiens
  hsa-miR-766
acuccagccccacagccucagc

SEQ ID NO: 288 - RNA, 23 nt, Homo sapiens
  hsa-miR-770-5p
uccaguaccacgugucagggcca

SEQ ID NO: 289 - RNA, 23 nt, Homo sapiens
  hsa-miR-136
acuccauuuguuuugaugaugga

SEQ ID NO: 290 - RNA, 21 nt, Homo sapiens
  hsa-miR-640
augauccaggaaccugccucu

SEQ ID NO: 291 - RNA, 22 nt, Homo sapiens
  hsa-miR-30e-5p_v9.1
uguaaacauccuugacuggaag

SEQ ID NO: 292 - RNA, 22 nt, Homo sapiens
  hsa-miR-30a
uguaaacauccucgacuggaag

SEQ ID NO: 293 - RNA, 22 nt, Homo sapiens
  hsa-miR-409-3p
gaauguugcucggugaaccccu

SEQ ID NO: 294 - RNA, 23 nt, Homo sapiens
  hsa-miR-17-3p_v9.1
caaagugcuuacagugcagguag

SEQ ID NO: 295 - RNA, 22 nt, Homo sapiens
  hsa-miR-29a_v9.1
uagcaccaucugaaaucgguua

SEQ ID NO: 296 - RNA, 22 nt, Homo sapiens
  hsa-miR-130a
cagugcaauguuaaaagggcau

SEQ ID NO: 297 - RNA, 22 nt, Homo sapiens
  hsa-miR-194
uguaacagcaacuccaugugga

SEQ ID NO: 298 - RNA, 22 nt, Homo sapiens
  hsa-miR-338_v9.1
uccagcaucagugauuuuguug

SEQ ID NO: 299 - RNA, 21 nt, Homo sapiens
  hsa-miR-525-5p
cuccagagggaugcacuuucu

SEQ ID NO: 300 - RNA, 22 nt, Homo sapiens
  hsa-miR-92b_v9.1
uauugcacucgucccggccucc

SEQ ID NO: 301 - RNA, 21 nt, Homo sapiens
  hsa-miR-23b
aucacauugccagggauuacc

SEQ ID NO: 302 - RNA, 21 nt, Homo sapiens
  hsa-miR-652_v9.1
aauggcgccacuaggguugug

SEQ ID NO: 303 - RNA, 22 nt, Homo sapiens
  hsa-miR-491_v9.1
aguggggaacccuuccaugagg

SEQ ID NO: 304 - RNA, 23 nt, Homo sapiens
  hsa-miR-623
aucccuugcaggggcuguugggu

SEQ ID NO: 305 - RNA, 21 nt, Homo sapiens
  hsa-miR-331-3p
gccccugggccuauccuagaa

SEQ ID NO: 306 - RNA, 23 nt, Homo sapiens
  hsa-miR-346
ugucugcccgcaugccugccucu

SEQ ID NO: 307 - RNA, 22 nt, Homo sapiens
  hsa-miR-574_v9.1
cacgcucaugcacacacccaca

SEQ ID NO: 308 - RNA, 23 nt, Homo sapiens
  hsa-miR-614
gaacgccuguucuugccaggugg

SEQ ID NO: 309 - RNA, 22 nt, Homo sapiens
  hsa-miR-214_v9.1
acagcaggcacagacaggcagu

SEQ ID NO: 310 - RNA, 23 nt, Homo sapiens
  hsa-miR-145_v9.1
guccaguuuucccaggaaucccu

SEQ ID NO: 311 - RNA, 19 nt, Homo sapiens
  hsa-miR-648
aagugugcagggcacuggu

SEQ ID NO: 312 - RNA, 23 nt, Homo sapiens
  hsa-miR-371-3p
aagugccgccaucuuuugagugu

SEQ ID NO: 313 - RNA, 22 nt, Homo sapiens
  hsa-miR-22
aagcugccaguugaagaacugu

SEQ ID NO: 314 - RNA, 23 nt, Homo sapiens
  hsa-miR-199a-5p
cccaguguucagacuaccuguuc

SEQ ID NO: 315 - RNA, 22 nt, Homo sapiens
  hsa-miR-504_v9.1
agacccuggucugcacucuauc

SEQ ID NO: 316 - RNA, 19 nt, Homo sapiens
  hsa-miR-564
aggcacggugucagcaggc

SEQ ID NO: 317 - RNA, 21 nt, Homo sapiens
  hsa-miR-497
cagcagcacacugugguuugu

SEQ ID NO: 318 - RNA, 23 nt, Homo sapiens
  hsa-miR-557
guuugcacgggugggccuugucu

SEQ ID NO: 319 - RNA, 22 nt, Homo sapiens
  hsa-miR-320_v9.1
aaaagcuggguugagagggcga

SEQ ID NO: 320 - RNA, 22 nt, Homo sapiens
  hsa-miR-21
uagcuuaucagacugauguuga

SEQ ID NO: 321 - RNA, 22 nt, Homo sapiens
  hsa-miR-486-5p
uccuguacugagcugccccgag

SEQ ID NO: 322 - RNA, 22 nt, Homo sapiens
  hsa-miR-146a
ugagaacugaauuccauggguu

SEQ ID NO: 323 - RNA, 20 nt, Homo sapiens
  hsa-miR-620
auggagauagauauagaaau

SEQ ID NO: 324 - RNA, 22 nt, Homo sapiens
  hsa-miR-186_v9.1
caaagaauucuccuuuugggcu

SEQ ID NO: 325 - RNA, 22 nt, Homo sapiens
  hsa-miR-375
uuuguucguucggcucgcguga

SEQ ID NO: 326 - RNA, 23 nt, Homo sapiens
  hsa-miR-671_v9.1
aggaagcccuggaggggcuggag

SEQ ID NO: 327 - RNA, 22 nt, Homo sapiens
  hsa-miR-617
agacuucccauuugaagguggc

SEQ ID NO: 328 - RNA, 21 nt, Homo sapiens
  hsa-miR-376a
aucauagaggaaaauccacgu

SEQ ID NO: 329 - RNA, 23 nt, Homo sapiens
  hsa-miR-542-5p_v9.1
ucggggaucaucaugucacgaga

SEQ ID NO: 330 - RNA, 21 nt, Homo sapiens
  hsa-miR-143_v9.1
ugagaugaagcacuguagcuc

SEQ ID NO: 331 - RNA, 21 nt, Homo sapiens
  hsa-miR-662
ucccacguuguggcccagcag

SEQ ID NO: 332 - RNA, 22 nt, Homo sapiens
  hsa-miR-223_v9.1
ugucaguuugucaaauacccca

SEQ ID NO: 333 - RNA, 23 nt, Homo sapiens
  hsa-miR-330-3p
gcaaagcacacggccugcagaga

SEQ ID NO: 334 - RNA, 21 nt, Homo sapiens
  hsa-miR-33_v9.1
gugcauuguaguugcauugca

SEQ ID NO: 335 - RNA, 21 nt, Homo sapiens
  hsa-miR-502-5p
auccuugcuaucugggugcua

SEQ ID NO: 336 - RNA, 23 nt, Homo sapiens
  hsa-miR-339_v9.1
ucccuguccuccaggagcucacg

SEQ ID NO: 337 - RNA, 22 nt, Homo sapiens
  hsa-miR-627
gugagucucuaagaaaagagga

SEQ ID NO: 338 - RNA, 22 nt, Homo sapiens
  hsa-miR-197
uucaccaccuucuccacccagc

SEQ ID NO: 339 - RNA, 22 nt, Homo sapiens
  hsa-miR-29c_v9.1
uagcaccauuugaaaucgguua

SEQ ID NO: 340 - RNA, 22 nt, Homo sapiens
  hsa-miR-584
uuaugguuugccugggacugag

SEQ ID NO: 341 - RNA, 22 nt, Homo sapiens
  hsa-miR-199a*_v9.1
acaguagucugcacauugguua

SEQ ID NO: 342 - RNA, 22 nt, Homo sapiens
  hsa-miR-452*_v9.1
cucaucugcaaagaaguaagug

SEQ ID NO: 343 - RNA, 22 nt, Homo sapiens
  hsa-miR-181c
aacauucaaccugucggugagu

SEQ ID NO: 344 - RNA, 21 nt, Homo sapiens
  hsa-miR-142-5p_v9.1
cauaaaguagaaagcacuacu

SEQ ID NO: 345 - RNA, 22 nt, Homo sapiens
  hsa-miR-148a
ucagugcacuacagaacuuugu

SEQ ID NO: 346 - RNA, 21 nt, Homo sapiens
  hsa-miR-631
agaccuggcccagaccucagc

SEQ ID NO: 347 - RNA, 22 nt, Homo sapiens
  hsa-miR-517*
ccucuagauggaagcacugucu

SEQ ID NO: 348 - RNA, 22 nt, Homo sapiens
  hsa-miR-452_v9.1
aacuguuugcagaggaaacuga

SEQ ID NO: 349 - RNA, 23 nt, Homo sapiens
  hsa-miR-518c*
ucucuggagggaagcacuuucug

SEQ ID NO: 350 - RNA, 19 nt, Homo sapiens
  hsa-miR-566
gggcgccugugaucccaac

SEQ ID NO: 351 - RNA, 22 nt, Homo sapiens
  hsa-miR-484
ucaggcucaguccccucccgau

SEQ ID NO: 352 - RNA, 23 nt, Homo sapiens
  hsa-miR-103
agcagcauuguacagggcuauga

SEQ ID NO: 353 - RNA, 22 nt, Homo sapiens
  hsa-miR-424
cagcagcaauucauguuuugaa

SEQ ID NO: 354 - RNA, 22 nt, Homo sapiens
  hsa-miR-510_v9.1
uacucaggagaguggcaaucac

SEQ ID NO: 355 - RNA, 22 nt, Homo sapiens
  hsa-miR-769-5p
ugagaccucuggguucugagcu

SEQ ID NO: 356 - RNA, 22 nt, Homo sapiens
  hsa-miR-140-5p
cagugguuuuacccuaugguag

SEQ ID NO: 357 - RNA, 22 nt, Homo sapiens
  hsa-miR-494_v9.1
ugaaacauacacgggaaaccuc

SEQ ID NO: 358 - RNA, 22 nt, Homo sapiens
  hsa-miR-377
aucacacaaaggcaacuuuugu

SEQ ID NO: 359 - RNA, 23 nt, Homo sapiens
  hsa-miR-18b_v9.1
uaaggugcaucuagugcaguuag

SEQ ID NO: 360 - RNA, 23 nt, Homo sapiens
  hsa-miR-425-3p_v9.1
aaugacacgaucacucccguuga

SEQ ID NO: 361 - RNA, 20 nt, Homo sapiens
  hsa-miR-324-3p
acugccccaggugcugcugg

SEQ ID NO: 362 - RNA, 23 nt, Homo sapiens
  hsa-miR-10a
uacccuguagauccgaauuugug

SEQ ID NO: 363 - RNA, 22 nt, Homo sapiens
  hsa-miR-26a_v9.1
uucaaguaauccaggauaggcu

SEQ ID NO: 364 - RNA, 22 nt, Homo sapiens
  hsa-miR-139_v9.1
ucuacagugcacgugucuccag

SEQ ID NO: 365 - RNA, 23 nt, Homo sapiens
  hsa-miR-181b_v9.1
aacauucauugcugucggugggu

(19) United States
(12) Patent Application Publication
(10) Pub. No.: US 2014/0031246 A1
(43) Pub. Date: Jan. 30, 2014

(54) CIRCULATING MICRORNAS ARE BIOMARKERS OF VARIOUS DISEASES

(71) Applicants: Stephen J. Meltzer, Lutherville, MD (US); Jee-Hoon Song, Baltimore, MD (US); Yulan Cheng, Ellicott City, MD (US)

(72) Inventors: Stephen J. Meltzer, Lutherville, MD (US); Jee-Hoon Song, Baltimore, MD (US); Yulan Cheng, Ellicott City, MD (US)

(73) Assignee: THE JOHNS HOPKINS UNIVERSITY, Baltimore, MD (US)

(21) Appl. No.: 13/811,530

(22) PCT Filed: Jul. 22, 2011
(86) PCT No.: PCT/US2011/044963

§ 371 Date: Aug. 12, 2013

Related U.S. Application Data
(60) Provisional application No. 61/366,596, filed on Jul. 22, 2010.

(51) Int. Cl. C12Q 1/68

(52) U.S. Cl. CPC C12Q 1/6886
USPC 506/9; 435/6.11; 536/24.31; 536/24.33; 506/16; 435/6.12

(57) ABSTRACT

The present invention relates to the field of biomarkers. More specifically, the present invention relates to the use of biomarkers to diagnose and monitor various diseases such as cancer.

### CROSS-REFERENCE TO RELATED APPLICATIONS

This application claims the benefit of U.S. Provisional Application No. 61/366,596, filed Jul. 22, 2010; which is incorporated herein by reference in its entirety.

### STATEMENT OF GOVERNMENTAL INTEREST

This invention n was made with U.S. government support under grant no. NIH-CA133012. The U.S. government has certain rights in the invention.

### FIELD OF THE INVENTION

The present intention relates to the field of biomarkers. More specifically, the present invention relates to the use of biomarkers to diagnose at d monitor various diseases such as cancer.

### INCORPORATION-BY-REFERENCE OF MATERIAL SUBMITTED ELECTRONICALLY

This application contains a sequence listing. It has been submitted electronically via EFS-Web as an ASCII text file entitled “P11164-02.txt.” The sequence listing is 63.2 kilobytes in size, and was created on Jul. 20, 2011. It is hereby incorporated by reference in its entirety.

### BACKGROUND OF THE INVENTION

Esophageal adenocarcinoma (EAC) is one of the most rapidly increasing cancers in the United States and throughout the developed world. The current primary screening method for EAC is gastrointestinal endoscopy; however, this method is unsuitable and impractical for population-based screening or detection of asymptomatic EAC. Developing a test using biomarkers of EAC discovered in blood would be a cheaper, less invasive, and more robust alternative.

### SUMMARY OF THE INVENTION

The present invention relates to the field of biomarkers. More specifically, the present invention relates to the use of microRNA biomarkers to diagnose and monitor various diseases such as cancer. The present invention is based, at least in part, on the discovery of that microRNA can serve as biomarkers for various disease, specifically, EAC.

Accordingly, in one aspect, the present invention provides methods useful for diagnosing cancer, specifically, EAC. In one embodiment, the method for diagnosing espohageal adenocarcinoma (EAC) in a patient comprises the steps of (a) measuring the levels of at least one microRNA (miR) biomarker in a sample collected from the patient; and (b) comparing the levels of the at least one miR biomarker with corresponding levels in a control sample from a subject that does not have EAC, wherein a significant difference in the levels of the at least one miR biomarker in the sample from the patient and the corresponding levels in the control sample is indicative that the patient has EAC.

In a specific embodiment, the at least one miR biomarker is selected from the group consisting of hsa-miR-345, hsa-miR-663, hsa-miR-373*, and hsa-miR-630. In an alternative embodiment, the at least one miR biomarker comprises hsa-miR-345, hsa-miR-663, hsa-miR-373*m and hsa-miR-630. The at least one miR biomarker can further comprise one or more biomarkers selected from the group consisting of hsa-miR-193a, hsa-miR-328, hsa-miR-629.

In another embodiment, the at least one miR biomarker comprises hsa-miR-345, hsa-miR-663, hsa-miR-373*, hsa-miR-630, hsa-miR-193a, hsa-miR-328, and hsa-miR-629. In yet another embodiment, the at least one miR biomarker comprises hsa-miR-625, hsa-miR-25, hsa-miR-93, hsa-miR-106b, hsa-miR-155, hsa-miR-345, hsa-miR-130b, hsa-miR-429, hsa-miR-28, hsa-miR-30a-5-, hsa-miR-200a, and hsa-miR-765. In a further embodiment, the at least one miR biomarker comprises hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b. Alternatively, the at least one miR biomarker comprises hsa-miR-519e, hsa-miR-520a-5p, hsa-miR-181a*, hsa-miR-769-3p, hsa-miR-373, hsa-miR-191*, hsa-miR-195, hsa-miR-634, hsa-miR-765, hsa-miR-421, hsa-miR-518d-3p, hsa-miR-630, hsa-miR-92b_v9.1, hsa-miR-659, hsa-miR-663, hsa-miR-770-5p, hsa-miR-508-3p, hsa-miR-219-5p, hsa-miR-200a, hsa-miR-646, hsa-miR-345, hsa-miR-548-3p, and hsa-miR-380*.

In another embodiment, the at least one miR biomarker comprises hsa-miR-630, hsa-miR-345, hsa-miR-663, hsa-miR-328, hsa-miR-769-3p, hsa-miR-572, hsa-miR-622, hsa-miR-584, hsa-miR-625, hsa-miR-93, hsa-miR-155, hsa-miR-103b, hsa-miR-429, hsa-miR-186, hsa-miR-590, hsa-miR-339, hsa-miR-151, hsa-miR-28, hsa-miR-30a-5p, hsa-miR-200a, hsa-miR-301a, hsa-miR-15b, and hsa-miR-765. The at least one miR biomarker can further comprise one or more miR biomarkers selected form the group consisting of hsa-miR-223, hsa-miR-22, hsa-miR-575, hsa-miR-92, hsa-miR-21, hsa-miR-30d, hsa-miR-19b, hsa-miR-24, hsa-miR-25, hsa-miR-106b, hsa-miR-494, hsa-miR-197, hsa-miR-185, hsa-miR-138, hsa-miR-130a, hsa-miR-377, hsa-miR-34c, hsa-miR-100, hsa-miR-125b, hsa-miR-136, hsa-miR-125a, hsa-miR-34b, hsa-miR-503, hsa-miR-203, hsa-miR-193a, hsa-miR-146a, hsa-miR-574, and hsa-miR-27a.

In a specific embodiment, the sample is a serum sample. In another embodiment, the sample is a plasma sample. In an alternative embodiment, the sample is a blood sample.

A method for diagnosing esophageal adenocarcinoma (EAC) in a patient can also comprise the steps of (a) measuring the levels of a panel of microRNA (miR) biomarkers in a sample collected from the patient, wherein panel comprises hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b; and (b) comparing the levels of the panel or miR biomarkers with corresponding levels in a control sample from a subject that does not have EAC, wherein a significant difference in the levels of the panel or miR biomarkers in the sample from the patient and the corresponding levels in the control sample is indicative that the patient has EAC. In a specific embodiment, the sample is a blood, serum or plasma sample.

The present invention also provides methods for determining the cancer status in a subject. In a specific embodiment, the method comprises (a) measuring the levels of at least one biomarker in a sample collected from the subject; and (b) comparing the levels of the at least one biomarker with the corresponding levels in a control sample from a subject that does not have cancer and the corresponding levels in a control sample from a subject that has EAC, wherein a correlation between the levels of the at least one biomarker in the sample from the subject and the corresponding levels in one of the control samples is determinative of the cancer status of the subject. In a more specific embodiment, the at least one biomarker measured comprises hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b. In one embodiment, the sample is a blood, serum or plasma sample. In a specific embodiment, the cancer is esophageal adenocarcinoma (EAC). In a particular embodiment, the stage of EAC is determined. In another embodiment, the progression of EAC is determined.

In another aspect, the present invention provides kits useful for diagnosing EAC. In a specific embodiment, a kit comprises miR hybridization or amplification reagents comprising one or more probe or amplification primers for hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b.

In other embodiments, the kit comprises miR hybridization or amplification reagents comprising one or more probe or amplification primers for a panel of miR biomarkers. In a specific embodiment, the panel or miR biomarkers comprises one or more miR biomarkers selected from the group consisting of hsa-miR-345, hsa-miR-663, hsa-miR-373*, and hsa-miR-630. Alternatively, the panel of miR biomarkers comprises hsa-miR-345, hsa-miR-663, hsa-miR-373*, and hsa-miR-630. The panel of miR biomarkers can further comprise one or more biomarkers selected from the group consisting of hsa-miR-193a, hsa-miR-328, hsa-miR-629.

In another embodiment, the panel or miR biomarkers comprises hsa-miR-345, hsa-miR-663, hsa-miR-373*, hsa-miR-630, hsa-miR-193a, hsa-miR-328, and hsa-miR-629. In yet another embodiment, the panel of miR biomarkers comprises hsa-miR-625, hsa-miR-25, hsa-miR-93, hsa-miR-106b, hsa-miR-155, hsa-miR-345, hsa-miR-130b, hsa-miR-429, hsa-miR-28, hsa-miR-30a-5p, hsa-miR-200a, and hsa-miR-765. In a further embodiment, the panel of miR biomarkers comprises hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b. In still a further embodiment, the panel of miR biomarkers comprises hsa-miR-519e, hsa-miR-520a-5p, hsa-miR-181a*, hsa-miR-769-3p, hsa-miR-373, hsa-miR-191*, hsa-miR-195, hsa-miR-634, hsa-miR-765, hsa-miR-421, hsa-miR-518d-3p, hsa-miR-630, hsa-miR-92b_{—v}9.1, hsa-miR-659, hsa-miR-663, hsa-miR-770-5p, hsa-miR-508-3p, hsa-miR-219-5p, hsa-miR-200a, hsa-miR-646, hsa-miR-345, hsa-miR-548d-3p, and hsa-miR-380*.

In a specific embodiment, the panel of miR biomarkers comprises hsa-miR-630, hsa-miR-345, hsa-miR-663, hsa-miR-328, hsa-miR-769-3p, hsa-miR-572, hsa-miR-622, hsa-miR-584, hsa-miR-625, hsa-miR-93, hsa-miR-155, hsa-miR-130b, hsa-miR-429, hsa-miR-186, hsa-miR-590, hsa-miR-339, hsa-miR-151, hsa-miR-28, hsa-miR-30a-5p, hsa-miR-200a, hsa-miR-301a, hsa-miR-15b, and hsa-miR-765. The panel of miR biomarkers can further comprise one or more miR biomarkers selected from the group consisting of hsa-miR-223, hsa-miR-22, hsa-miR-575, hsa-miR-92, hsa-miR-21, hsa-miR-30d, hsa-miR-19b, hsa-miR-24, hsa-miR-25, hsa-miR-106b, hsa-miR-494, hsa-miR-197, hsa-miR-185, hsa-miR-138, hsa-miR-130a, hsa-miR-377, hsa-miR-34c, hsa-miR-100, hsa-miR-125b, hsa-miR-136, hsa-miR-125a, hsa-miR-34b, hsa-miR-503, hsa-miR-203, hsa-miR-193a, hsa-miR-146a, hsa-miR-574, and hsa-miR-27a.

In particular embodiments, the miR biomarker panel comprises one or more of hsa-miR-30a*, hsa-miR-206, hsa-miR-513_v9.1, hsa-miR-583, hsa-miR-629*, hsa-miR-515-3p_v9.1, hsa-miR-571, hsa-miR-33b_v9.1, hsa-miR-125b, hsa-miR-522_v9.1, hsa-miR-199b-5p, hsa-miR-191_v9.1, hsa-miR-9, hsa-miR-1_v9.1, hsa-miR-660, hsa-miR-765, hsa-miR-146b-5p, hsa-miR-135b_v1.9, hsa-miR-523_v9.1, hsa-miR-381, hsa-miR-490-3p, hsa-miR-210, hsa-miR-200a, hsa-miR-520b, hsa-miR-147, hsa-miR-16, hsa-miR-591, hsa-miR-668, hsa-miR-101_v9.1, hsa-miR-519b_v9.1, hsa-miR-128b_v1.9, hsa-miR-650, hsa-miR-518d-3p, hsa-miR-423_v9.1, hsa-miR-19b, hsa-miR-586, hsa-miR-639, hsa-miR-196a_v9.1, hsa-miR-187_v9.1, hsa-miR-520f_v1.9, hsa-miR-144_v9.1, hsa-miR-767-5p, hsa-miR-592, hsa-miR-492, hsa-miR-95, hsa-miR-422b_v9.1, hsa-miR-526a_v9.1, hsa-miR-34b*, hsa-miR-302b, hsa-miR-602, hsa-miR-15b, hsa-miR-30c, hsa-miR-585, hsa-miR-493-3p_v9.1, hsa-miR-518a-3p, hsa-miR-550*, hsa-miR-483_v9.1, hsa-miR-17-5p_v9.1, hsa-miR-500*, hsa-miR-520e, hsa-miR-520g, hsa-miR-615_v9.1, hsa-miR-26b_v9.1, hsa-miR-449a, hsa-miR-499_v9.1, hsa-miR-196b_v9.1, hsa-miR-203, hsa-miR-548d-3p, hsa-miR-548b-3p, hsa-miR-126_v9.1, hsa-miR-518c_v9.1, hsa-miR-337_v9.1, hsa-miR-605, hsa-miR-551b, hsa-miR-92_v9.1, hsa-miR-224_v9.1, hsa-miR-645, hsa-miR-190, hsa-miR-106a_v9.1, hsa-miR-517b, hsa-miR-185_v9.1, hsa-let-7a, hsa-miR-129-5p, hsa-miR-299-3p, hsa-miR-25, hsa-miR-605, hsa-miR-498, hsa-miR-449b, hsa-miR-302a*_v9.1, hsa-miR-613, hsa-miR-519d_v9.1, hsa-miR-425, hsa-miR-328, hsa-miR-619, hsa-miR-380, hsa-miR-507, hsa-miR-302d, hsa-miR-630, hsa-miR-433, hsa-miR-493*, hsa-let7f, hsa-miR-184, hsa-miR-422a_v9.1, hsa-miR-587, hsa-miR-296-5p, hsa-miR-573, hsa-miR-192, hsa-miR-556_v9.1, hsa-miR-374a, hsa-miR-107, hsa-miR-659, hsa-miR-628_v9.1, hsa-miR-609, hsa-miR-18a*_v9.1, hsa-miR-485-3p, hsa-miR-141, hsa-miR-519c-3p, hsa-miR-132, hsa-miR-624*, hsa-miR-20a, hsa-miR-421, hsa-let-7e_v9.1, hsa-miR-212, hsa-miR-20b, hsa-miR-576_v9.1, hsa-miR-526b_v9.1, hsa-miR-649, hsa-miR-519a_v9.1, hsa-miR-100,hsa-miR-607, hsa-miR-643, hsa-let-7g_v9.1, hsa-miR-98, hsa-miR-195, hsa-miR-603, hsa-miR-448, hsa-miR-34a_v9.1, hsa-miR-539, hsa-miR-520d*_v9.1, hsa-miR-568, hsa-miR-299-5p, hsa-miR-34c-5p, hsa-miR-152,v9.1, hsa-miR-553, hsa-miR-516a-3p, hsa-miR-122a_v9.1, hsa-miR-644, hsa-miR-599, hsa-miR-514_v9.1, hsa-miR-512-3p, hsa-miR-578, hsa-miR-561, hsa-miR-149_v 9.1, hsa-miR-625_v9.1, hsa-miR-134v9.1, hsa-miR-454-3p_v9.1, h sa-mi R-373*, hsa-miR-198_v9.1, hsa-miR-610, hsa-miR-658, hsa-miR-383, hsa-miR-30b, hsa-miR-663, hsa-miR-552, hsa-miR-519e_v9.1, hsa-miR-453_v9.1, hsa-miR-29b, hsa-miR-127-3p, hsa-miR-367, hsa-miR-545_v9.1, hsa-miR-532-5p, hsa-miR-32_v9.1, hsa-miR-516b, hsa-miR-200b_v9.1, hsa-miR-595, hsa-miR-362-5p, hsa-miR-99a, hsa-miR-601, hsa-miR-191*, hsa-miR-128a_v9.1, hsa-miR-651, hsa-miR590-5p, hsa-miR-501-5p, hsa-miR-526b*, hsa-miR-487b, hsa-miR-608, hsa-miR-508-3p, hsa-miR-19a, hsa-let-7i_v9.1, hsa-miR-518f*, hsa-miR-181d_v9.1, hsa-miR-302c*, hsa-miR-373, hsa-miR-517c, hsa-let-7b, hsa-miR-769-3p, hsa-miR-655, hsa-miR-432, hsa-miR-325, hsa-miR-135a, hsa-miR-7_v9.1, hsa-miR-193a_v9.1, hsa -miR27b, hsa-miR-202_v9.1, hsa-let-7d_v9.1, hsa-miR-562, hsa-miR-183_v9.1, hsa-miR-596, hsa-miR-622, hsa-miR-370_v9.1, hsa-miR-155_v9.1, hsa-miR-302c, hsa-miR-524-3p, hsa-miR-363, hsa-miR-582-5p, hsa-miR-301a, hsa-miR-138_v9.1, hsa-miR-520d_v9.1, hsa-miR-518f_v9.1, hsa-miR-28-5p, hsa-let-7c, hsa-miR-369-5p, hsa-miR-450_v9.1, hsa-miR-137, hsa-miR-524-5p, hsa-miR-520h, hsa-miR-572, hsa-miR-495_v9.1, hsa-miR-758, hsa-miR-10b_v9.1, hsa-miR-580, hsa-miR-215, hsa-miR-378*, hsa-miR-30e*, hsa-miR-93, hsa-miR-521, hsa-miR-511, hsa-miR-379_v9.1, hsa-miR-431, hsa-miR-382, hsa-miR-632, hsa-miR-647, hsa-miR-182_v9.1, hsa-miR-154, hsa-miR188_v9.1, hsa-miR-520a-3p, hsa-miR-302a, hsa-miR-451_v9.1, hsa-miR-133a_v9.1, hsa-miR-126*, hsa-miR-24, hsa-miR-30d, hsa-miR-221, hsa-miR-125a_v9.1, hsa-miR-335, hsa-miR-342_v9.1, hsa-miR-324-5p, hsa-miR-18a_v9.1, hsa-miR-181a, hsa-miR-575, hsa-miR-365, hsa-miR-27a, hsa-miR-130b, hsa-miR-193b_v9.1, hsa-miR-222_v9.1, hsa-miR-200c_v9.1, hsa-miR-106b, hsa-miR-361-5p, hsa-miR-326, hsa-miR-148b, hsa-miR-151-3p, hsa-miR-410, hsa-527_v9.1, hsa-miR-205, hsa-miR-99b, hsa-miR-15a, hsa-miR-23a, hsa-miR-636_v9.1, hsa-miR-638, hsa-miR-345_v9. 1, hsa-miR-519e*, hsa-miR-654-5p, hsa-miR-142-3p, hsa-miR-31_v9.1, hsa-miR-133b, hsa-miR-766, hsa-miR-770-5p, hsa-miR-130, hsa-miR-640, hsa-miR-30e-5p_v9.1, hsa-miR-30a, hsa-miR-409-3p, hsa-miR-17-3p_v9. 1, hsa-miR-29a_v9.1, hsa-miR-130a, hsa-miR-194, hsa-miR-338_v9.1, hsa-miR-525-5p, hsa-miR-92b_v9. 1, hsa-miR-23b, hsa-miR-652_v9.1, hsa-miR-491_v9.1, hsa-miR-623, hsa-miR-331-3p, hsa-miR-346, hsa-miR-574 _v9.1, hsa-miR-614, hsa-miR-214_v9.1, hsa-miR-145_v9.1, hsa-miR-648, hsa-miR-371-3p, hsa-miR-22, hsa-miR-199a-5p, hsa-miR-504_v9.1, hsa-miR-564, hsa-miR-497, hsa-miR-557, hsa-miR-320_v9.1, hsa-miR-21, hsa-miR-486-5p, hsa-miR-146a, hsa-miR-620, hsa-miR-186_v9.1, hsa-miR-375, hsa-miR-671_v9.1, hsa-miR-617, hsa-miR-376a, hsa-miR-542-5p_v9.1, hsa-miR-143_v9.1, hsa-miR-662, hsa-miR-223_v9. 1, hsa-miR-330-3p, hsa-miR-33_v9.1, hsa-miR-502-5p, hsa-miR-339_v9.1, hsa-miR-627, hsa-miR-197, hsa-miR-29c_v9.1, hsa-miR-584, hsa-miR-199a*_v9.1, hsa-miR-452*_v9.1, hsa-miR-181c, hsa-miR-142-5p_v9.1, hsa-miR-148a, hsa-miR-631, hsa-miR-517*, hsa-miR-452_v9.1, hsa-miR-518c*, hsa-miR-566, hsa-miR-484, hsa-miR-103, hsa-miR-424, hsa-miR-510_v9.1, hsa-miR-769-5p, hsa-miR-140-5p, hsa-miR-494_v9.1, hsa-miR-377, hsa-miR-18b_v9.1, hsa-miR-425-3p_v9.1. hsa-miR-324-3p, hsa-miR-10a, hsa-miR-26a_v9.1, hsa-miR-139_v9.1, hsa-miR-181b_v9.1.

In other embodiment, the miR biomarkers of the present invention comprise one or more of the following upregulated (i.e., elevated levels in EAC versus normal) biomarkers: hsa-miR-30a*, hsa-miR-206, hsa-miR-513_v9.1, hsa-miR-583, hsa-miR-629*, hsa-miR-515-3p_v9.1, hsa-miR-571, hsa-miR-33b_v9.1, hsa-miR-125b, hsa-miR-522_v9.1, hsa-miR-199b-5p, hsa-miR-191_v9.1, hsa-miR-9, hsa-miR-1_v9.1, hsa-miR-660, hsa-miR-765, hsa-miR-146b-5p, hsa-miR-135b_v9.1, hsa-miR-523_v9.1, hsa-miR-381, hsa-miR-490-3p, hsa-miR-210, hsa-miR-200a, hsa-miR-520b, hsa-miR-147, hsa-miR-16, hsa-miR-591, hsa-miR-668, hsa-miR-101_v9.1 hsa-miR-519b_v9.1, hsa-miR-128b_v9.1, hsa-miR-650, hsa-miR-518d-3p, hsa-miR-423_v9.1, hsa-miR-19b, hsa-miR-586, hsa-miR-639, hsa-miR-196a_v9.1. hsa-miR-187_v9.1, hsa-miR-520f_v9.1, hsa-miR-144_v9.1, hsa-miR-767-5p, hsa-miR-592, hsa-miR-492, hsa-miR-95, hsa-miR-422b_v9.1, hsa-miR-526a_v9.1, hsa-miR-34b*, hsa-miR-302b, hsa-miR-602, hsa-miR-15b, hsa-miR-30c, hsa-miR-585, hsa-miR-493-3p_v9.1, hsa-miR-518a-3p, hsa-miR-550*, hsa-miR-483_v9.1, hsa-miR-17-5p_v9.1, hsa-miR-500*, hsa-miR-520e, hsa-miR-520g, hsa-miR-615_v9.1, hsa-miR-26b_v9.1, hsa-miR-449a, hsa-miR-499_v9.1, hsa-miR-196b_v9.1, hsa-miR-203, hsa-miR-548d-3p, hsa-miR-548b-3p, hsa-miR-126_v9.1, hsa-miR-518c_v9.1, hsa-miR-337_v9.1, hsa-miR-605, hsa-miR-551b, hsa-miR-92_v9.1, hsa-miR-224_v9.1, hsa-miR-645, hsa-miR-190, hsa-miR-106a_v9.1, hsa-miR-517b, hsa-miR-185_v9.1, hsa-let-7a, hsa-miR-129-5p, hsa-miR-299-3p, hsa-miR-25, hsa-miR-604, hsa-miR-498, hsa-miR-449b, hsa-miR-302a*_v9.1, hsa-miR-613, hsa-miR-519d_v9.1, hsa-miR-425, hsa-miR-328, hsa-miR-619, hsa-miR-380, hsa-miR-507, hsa-miR-302d, hsa-miR-630, hsa-miR-433, hsa-miR-493*, hsa-let-7f, hsa-miR-184, hsa-miR-422a_v9.1, hsa-miR-587, hsa-miR-296-5p, hsa-miR-573, hsa-miR-192, hsa-miR-556_v9.1, hsa-miR-374a, hsa-miR-107, hsa -miR-659, hsa-miR-628_v9.1, hsa-miR-609, hsa-miR-18a*_v9.1, hsa-miR-485-3p, hsa-miR-141, hsa-miR-519c-3p, hsa-miR-132, hsa-miR-624*, hsa-miR-20a, hsa-miR-421, hsa-let-7e_v9.1, hsa-miR-212, hsa-miR-20b, hsa-miR-576_v9.1, hsa-miR-526b_v9.1, hsa-miR-649, hsa-miR-519a_v9.1, hsa-miR-100, hsa-miR-607, hsa-miR-643, hsa-let-7g_v9.1, hsa-miR-98, hsa-miR-195, hsa-miR-603, hsa-miR-448, hsa-miR-34a_v9.1, hsa-miR-539, hsa-miR-520d*_v9.1, hsa-miR-568, hsa-miR-299-5p, hsa-miR-34c-5p, hsa-miR-152_v9.1, hsa-miR-553, hsa-miR-509_v9.1, hsa-miR-516a-3p, hsa-miR-122a_v9.1, hsa-miR-644, hsa-miR-599, hsa-miR-514_v9.1, hsa-miR-512-3p, hsa-miR-578, hsa-miR-561, hsa-miR-149_v9.1, hsa-miR-625_v9.1, hsa-miR-134_v9.1, hsa-miR-454-3p_v9.1, hsa-miR-373*, hsa-miR-198_v9.1, hsa-miR-610, hsa-miR-658, hsa-miR-383, hsa-miR-30b, hsa-miR-663, hsa-miR-552, hsa-miR-519e_v9.1, hsa-miR-453_v9.1, hsa-miR-29b, hsa-miR-127-3p, hsa-miR-367, hsa-miR-545_v9.1, hsa-miR-532-5p, hsa-miR-32_v9.1, hsa-miR-516h, hsa-miR-200b_v9.1, hsa-miR-595, hsa-miR-362-5p, hsa-miR-99a, hsa-miR-601, hsa-miR-191*, hsa-miR-128a_v9.1, hsa-miR-651, hsa-miR-590-5p, hsa-miR-501-5p, hsa-miR-526b*, hsa-miR-487b, hsa-miR-608, hsa-miR-508-3p, hsa-miR-19a, hsa-let-7i_v9.1, hsa-miR-518f*, hsa-miR-181d_v9.1, hsa-miR-302c*, hsa-miR-373, hsa-miR-517c, hsa-let-7b, hsa-miR-769-3p, hsa-miR-655, hsa-miR-432, hsa-miR-325, hsa-miR-135a, hsa-miR-7_v9.1, hsa-miR-193a_v9.1, hsa-miR-27b, hsa-miR-202_v9.1, hsa-let-7d_v9.1, hsa-miR-562, hsa-miR-183_v9.1, hsa-miR-596, hsa-miR-622, hsa-miR-370_v9.1, hsa-miR-155_{———}v9.1, hsa-miR-302c, hsa-miR-524-3p, hsa-miR-363, hsa-miR-582-5p, hsa-miR-301a, hsa-miR-138_v9.1, hsa-miR-520d_v9.1, hsa-miR-518f_v9.1, hsa-miR-28-5p, hsa-let-7c, hsa-miR-369-5p, hsa-miR-450_v9.1, hsa-miR-137, hsa-miR-524-5p, hsa-miR-520h, hsa-miR-572, hsa-miR-495_v9.1, hsa-miR-758, hsa-miR-10b_v9.1, hsa-miR-580, hsa-miR-215, hsa-miR-378*, hsa-miR-30e*, hsa-miR-93, hsa-miR-521, hsa-miR-511, hsa-miR-379_v9.1, hsa-miR-431, hsa-miR-382, hsa-miR-632, hsa-miR-647, hsa-miR-182_v9.1, hsa-miR-154, hsa-miR-188_v9.1, hsa-miR-520a-3p, hsa-miR-302a, hsa-miR-133a_v9.1, and hsa-miR-126*.

In other embodiments, the miR biomarkers of the present invention comprise one or more of the following downregulated (i.e., reduced levels in EAC versus normal) biomarkers: hsa-miR-24, hsa-miR-30d, hsa-miR-221, hsa-miR-125a_v9.1, hsa-miR-335, hsa-miR-342_v9.1, hsa-miR-324-5p, hsa-miR-18a_v9.1, hsa-miR-181a, hsa-miR-575, hsa-miR-365, hsa-miR-27a, hsa-miR-130b, hsa-miR-193b_v9.1, hsa-miR-222_v9.1, hsa-miR-200c_v9.1, hsa-miR-106b, hsa-miR-361-5p, hsa-miR-326, hsa-miR-148b, hsa-miR-151-3p, hsa-miR-410, hsa-miR-527_v9.1, hsa-miR-205, hsa-miR-99b, hsa-miR-15a, hsa-miR-23a, hsa-miR-636_v9.1, hsa-miR-638, hsa-miR-345_v9.1, hsa-miR-519e*, hsa-miR-654-5p, hsa-miR-142-3p, hsa-miR-31_v9.1, hsa-miR-133b, hsa-miR-766, hsa-miR-770-5p, hsa-miR-136, hsa-miR-640, hsa-miR-30e-5p_v9.1, hsa-miR-30a, hsa-miR-409-3p, hsa-miR-17-3p_v9.1, hsa-miR-29a_v9.1, hsa-miR-130a, hsa-miR-194, hsa-miR-338_v9.1, hsa-miR-525-5p, hsa-miR-92b_v9.1, hsa-miR-23b, hsa-miR-652_v9.1, hsa-miR-491_v9.1, hsa-miR-623, hsa-miR-331-3p, hsa-miR-346, hsa-miR-574v9.1, hsa-miR-614, hsa-miR-214_v9.1, hsa-miR-145_v9.1, hsa-miR-648, hsa-miR-371-3p, hsa-miR-22, hsa-miR-199a-5p, hsa-miR-504_v9.1, hsa-miR-564, hsa-miR-497, hsa-miR-557, hsa-miR-320_v9.1, hsa-miR-21, hsa-miR-486-5p, hsa-miR-146a, hsa-miR-620, hsa-miR-186_v9.1, hsa-miR-375, hsa-miR-671_v9.1, hsa-miR-617, hsa-miR-376a, hsa-miR-542-5_v9.1, hsa-miR-143_v9.1, hsa-miR-662, hsa-miR-223_v9.1, hsa-miR-330-3p, hsa-miR-33_v9.1, hsa-miR-502-5p, hsa-miR-339_v9.1, hsa-miR-627, hsa-miR-197, hsa-miR-29c_v9.1, hsa-miR-584, hsa-miR-199a*_v9.1, hsa-miR-452*_v9.1, hsa-miR-181c, hsa-miR-142-5p_v9. 1, hsa-miR-148a, hsa-miR-631, hsa-miR-517*, hsa-miR-452_v9.1, hsa-miR-518c*, hsa-miR-566, hsa-miR-484, hsa-miR-103, hsa-miR-424, hsa-miR-510_v9.1, hsa-miR-769-5p, hsa-miR-140-5p, hsa-miR-494_v9.1, hsa-miR-377, hsa-miR-18b_v9.1, hsa-miR-425-3p_v9.1, hsa-miR-324-3p, hsa-miR-10a, hsa-miR-26a_v9.1, hsa-miR-139_v9.1, hsa-miR-181b_v9.1.

In other embodiments, the miR biomarkers of the present invention comprise one or more of hsa-miR-519e*, hsa-miR-527_v9.1, hsa-miR-671_v9.1, hsa-miR-638, hsa-miR-520a-5p, hsa-miR-181a*, hsa-miR-769-3p, hsa-miR-373*, hsa-miR-518c*, hsa-miR-617, hsa-miR-492, hsa-miR-557, hsa-miR-181b_v9.1, hsa-miR-489, hsa-miR-622, hsa-miR-525-5p, hsa-miR-518f*_v9.1, hsa-miR-520d*_v9.1, hsa-miR-516b, hsa-let-7e_v9.1, hsa-miR-454*, hsa-miR-544_v9.1, hsa-miR-191*, hsa-miR-195, hsa-miR-634, hsa-miR-765, hsa-miR-495_v9.1, hsa-miR-202_v9.1, hsa-miR-625_v9.1, hsa-miR-452_v9.1, hsa-miR-567, hsa-miR-526a_v9.1, hsa-miR-526b_v9.1, hsa-miR-378*, hsa-miR-302c*, hsa-miR-601, hsa-miR-155_v9.1, hsa-miR-149_v9.1, hsa-miR-370 v9.1, hsa-miR-583, hsa-miR182_v9.1, hsa-miR-182*,hsa-miR-636_v9.1, hsa-miR-593*, hsa-miR-184,hsa-miR-200c_v9.1, hsa-miR-597, hsa-miR-188_v9.1, hsa-miR-498, hsa-miR-600, hsa-miR-423_v9.1, hsa-miR-24-1*, hsa-miR-490-3p, hsa-miR-524-5p, hsa-miR-100, hsa-miR-302d, hsa-miR-550*, hsa-miR-421, hsa-miR-518d-3p, hsa-miR-576_v9.1, hsa-miR-610, hsa-miR-210, hsa-miR-589*, hsa-miR-363*, hsa-miR-630, hsa-miR-562, hsa-miR-211, hsa-miR-92b_v9.1, hsa-miR-659, hsa-miR-337_v9.1, hsa-miR-618, hsa-miR-525*_v9.1, hsa-miR-598, hsa-miR-9*, hsa-miR-518e_v9.1, hsa-miR-214_v9.1, hsa-miR-591, hsa-miR-216_v9.1, hsa-miR-663, hsa-miR-770-5p, hsa-miR-508-3p, hsa-miR-154*, hsa-miR-596, hsa-miR-623, hsa-miR-30c, hsa-miR-219-5p, hsa-miR-602, hsa-miR-619, hsa-miR-510_v9.1, hsa-miR-612, hsa-miR-768-3p_v11.0, hsa-miR-206, hsa-miR-200a, hsa-miR-646, hsa-miR-516a-3p, hsa-miR-518b, hsa-miR-371-3p, hsa-miR-656, hsa-miR-422a_v9.1, hsa-miR-512-5p, hsa-miR-548a-3p, hsa-miR-411, hsa-miR-323-3p, hsa-miR-521, hsa-miR-607, hsa-miR-153_v9.1, hsa-miR-563, hsa-miR-647, hsa-miR-653_v9.1, hsa-miR-212, hsa-miR-329, hsa-miR-501-5p, hsa-miR-181d_v9.1, hsa-miR-542-5p_v9.1, hsa-miR-650, hsa-miR-452*_v9.1, hsa-miR-432, hsa-miR-217_v9.1, hsa-miR-611, hsa-miR-616*, hsa-miR-345_v9.1, hsa-miR-548d-3p, hsa-miR-380*, hsa-miR-380, hsa-miR-518c_v9.1 hsa-miR-30a*, hsa-miR560_v9.1, hsa-miR-99a, hsa-miR-124a_v9.1 hsa-miR-515-5p, hsa-miR-296-5p, hsa-miR-30b, hsa-miR-335, hsa-miR-579_v9.1, hsa-miR-10a, hsa-miR-520b, hsa-miR-376a*_v9.1, hsa-miR-375, hsa-miR-587, hsa-miR-572, hsa-miR-126*, hsa-miR-205, hsa-miR-549, and hsa-miR-564.

In further embodiments, the miR biomarkers of the present invention comprise one or more of hsa-miR-630, hsa-miR-345, hsa-miR-663, hsa-miR-328, hsa-miR-769-3p, hsa-miR-572, hsa-miR-622, hsa-miR-584, hsa-miR-625, hsa-miR-93, hsa-miR-155, hsa-miR-30b, hsa-miR-429, hsa-miR-186, hsa-miR-590, hsa-miR-339, hsa-miR-151, hsa-miR-28, hsa-miR-30a-5p, hsa-miR-200a, hsa-miR-301a, hsa-miR-15b, hsa-miR-765, hsa-miR-223, hsa-miR-22, hsa-miR-575, hsa-miR-92, hsa-miR-21, hsa-miR-30d, hsa-miR-19b, hsa-miR-24, hsa-miR-25, hsa-miR-106b, hsa-miR-494, hsa-miR-197, hsa-miR-185, hsa-miR-138, hsa-miR-130a, hsa-miR-377, hsa-miR-34c, hsa-miR-100, hsa-miR-125b, hsa-miR-136, hsa-miR-125a, hsa-miR-34b, hsa-miR-503, hsa-miR-203, hsa-miR-93a, hsa-miR-146a, hsa-miR-574, and hsa-miR-27a.

In more specific embodiments, the miR biomarkers of the present invention comprise two or more of hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130 b, hsa-miR-30a, hsa-miR-301, and hsa-miR-15b.

In a specific embodiment, a method for characterizing EAC in a patient comprises the steps of (a) measuring the level of a miR in a serum sample, wherein the miR comprise one or more of hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b; and (b) determining whether the level of miR is elevated in the sample, thereby characterizing EAC in the patient. In other embodiments, the miR can comprise any of the panel of miR discolored herein.

In another embodiment, a method for diagnosing EAC in a patient comprises evaluating expression of one or more of invention comprise two or more of hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b, wherein a difference in the expression in the sample from the patient and expression in a normal sample or reference is indicative of EAC. In other embodiments, the miR can comprise any of the panel of miR disclosed herein.

### BRIEF DESCRIPTION OF THE FIGURES

FIG. 1 is a graph showing the validation of miR-345 serum expression level by real-time PCR. MicroRNA-345 expression level from individual samples (14 esophageal adenocarcinoma (EAC) and 12 normal serum) was measured and validated by real-time PCR. EAC serum samples are colored in red and normal serum samples are colored in blue. MiR expression levels are normalized to miR-16.

FIG. 2 is a graph showing the validation of miR-345 cell line expression level by real-time PCR. MiR expression levels are normalized to RNU6B.

FIG. 3 shows the receiver operating, characteristic (ROC) curve generated for miR-345. The area under the curve (AUC) is 0.814. Based on the curve, achieving both high sensitivity and specificity to differentiate patients with EAC from healthy controls seems to be difficult by using miR-345 alone.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must he noted that as used herein and in the appended claims, the singular forms “a,” “an,” and “the” include the plural reference unless the context clearly dictates otherwise. Thus, for example, a reference to a “biomarker” is a reference to one or more biomarkers, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

The following definitions are used throughout this specification. Other definitions are embedded within the specification for ease of reference.

As used herein, “comparing” refers to making an assessment of how the proportion, level or cellular localization of one or biomarkers in a sample from a patient relates to the proportion, level or cellular localization of the corresponding one or more biomarkers in a standard or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in standard or control sample.

As used herein, “indicates” or “correlates” (or “indication” or “correlation,” depending on the content) in reference to a parameter, e.g., a modulated proportion, level, or cellular localization in the cell from a patient, may mean that the patient has cancer. In specific embodiments, the parameter may comprise the presence, absence and/or particular amounts of one or more biomarkers of the present invention. A particular set or pattern of one or more biomarkers (including the presence, and/or particular amounts) may indicate that a patient has cancer (or correlated to a patient having cancer), in particular, EAC. In other embodiments, a particular set or pattern of one or more biomarkers (including the presence, absence, and/or particular amounts) may be correlated to a patient having Barrett's Esophagus (BE) (or may indicate that a patient has BE). In yet other embodiments, a particular set or pattern of one or more biomarkers (including the presence, absence, and/or particular amounts) may be correlated to a patient being unaffected. In certain embodiments, “correlating” or “normalization” as used according to the present invention may be by any method of relating levels of expression or localization of markers to a standard valuable for the: assessment of the diagnosis, prediction of a cancer or cancer progression, assessment of efficacy of clinical treatment, identification of a tumor that may respond to a treatment, selection of a patient for a particular treatment, monitoring of the progress of treatment, and in the context of a screening assay, for the identification of an anti-EAC therapeutic.

The terms “individual,” “subject” or “patient” are used interchangeably herein, and refer to a mammal, particularly, a human. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

The term “measuring” means methods which include detecting the presence or absence of a biomarker(s) in a sample, quantifying the amount of biomarker(s) in the sample, and/or qualifying the type of biomarker(s). Measuring can be accomplished by methods known in the art and those further described herein including, but not limited to, polymerase chain reaction. The term “measuring” is used interchangeably throughout with the term “detecting.”

Various methodologies of the instant invention include a step that involved comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes. In one embodiment, a “suitable control” or “appropriate control” is a value, level, feature, characteristic, property, etc. determined in a cell, organ, or patient, e.g., a control or normal cell, organ, or patient, exhibiting, for example, normal traits. For example, the biomarkers of the present invention may be assayed for their presence in a sample from an unaffected individual (UI) or a normal control individual (NC) (both terms are used interchangeably herein). In another embodiment, a “suitable control” or “appropriate control” is a value, level, feature, characteristic, property, etc. determined prior to performing a cancer therapy on a patient. In yet another embodiment, a transcription rate, mRNA level, translation rate, protein level, biological activity, cellular characteristic or property, genotype, phenotype, etc. can be determined prior to, during, or after administering a cancer therapy into a cell, organ, or patient. In a further embodiment, a “suitable control” or “appropriate control” is a predefined value, level, feature, characteristic, property, etc.

### II. MicroRNA Biomarkers for Esophageal Adenocarcinoma (EAC)

As used herein, the terms “microRNA,” “miRNA,” or “miR” are synonymous and include human miR, mature single stranded miR, precursor miR (pre-miR), and variants thereof. In some instances, the terms also include primary miR transcripts and duplex miR. The sequences for particular miR, including human mature and precursor sequences, can be found in several publicly available database including, but not limited to, the miRBase database (accessible at http://www.mirbase.org). For certain miR, a single precursor contains more than one mature miR sequence. In other instances, multiple precursor miR contain the same mature sequence. In some instances, mature miR have been renamed based on new scientific consensus. One of ordinary skill in the art appreciates that scientific consensus regarding the precise nucleic acid sequence for a given miR, in particular for mature forms of the miR, may change with time.

In one aspect, the present invention provides a panel of serum or plasma miR as biomarkers for EAC. In particular embodiments, miR that are present at elevated levels in the serum or plasma of patients with EAC are used as biomarkers. In other embodiments, miR that are present at reduced levels in the serum or plasma of patients with EAC are used as biomarkers. In some embodiments, more than one miR from serum or plasma can be used as biomarkers. In such cases, the miR may all have elevated levels, all have reduced levels, or a mixture of miR with elevated and reduced levels may be used.

The terms “reduced levels” or “elevated levels” refer to the amount of a miR in a serum or plasma sample from a patient compared to the amount of the miR in serum or plasma from a suitable control. For example, a miR present in the sera of an EAC patient may be determined to be present at lower amounts than in serum from a subject who does not have EAC. For certain miR, elevated levels in a patient serum or plasma sample correlate or indicate presence of or prognosis for EAC. Other miR are present in reduced levels in patients with EAC.

In particular embodiments, the level of the miR marker will be compared to a suitable control to determine whether the level is reduced or elevated. The control may be an external control, such as a miR in a serum or plasma sample from a patient known to be free of EAC. In other embodiments, the external control may be a miR from a non-serum sample like a tissue sample or a known amount of a synthetic RNA. An internal control may be a miR from the same serum or plasma sample being tested. The identity of a miR control may be the same as or different from the patient serum or plasma miR being measured.

The term “characterizing” includes making diagnostic or prognostic determinations or predictions of disease. In some instances, “characterizing” includes identifying whether a subject has a cancer such as EAC. The term “characterizing” further includes distinguishing patients with EAC from patients having other diseases. In other circumstances, “characterizing” includes determining the stage or aggressiveness of a disease state such as EAC, determining an appropriate treatment method for EAC, or assessing the effectiveness of a treatment for EAC.

The methods of the present invention can be used to characterize a patient with at least about 80%, at least about 85%, at least about 90%, at least about 91%, at least about 92%, at least about 93%, at least about 94%, at least about 95%, at least about 96%, at least about 97%, at least about 98%, or at least about 99% sensitivity. The degree of sensitivity indicates the percentage of patients with a disease who are positively characterized as having the disease. The methods described herein can also be used to characterize a patient with at least about 80%, at least about 85%, at least about 90%, at least about 91%, at least about 92%, at least about 93%, at least about 94%, at least about 95%, at least about 96%, at least about 97%, at least about 98%, or at least about 99% specificity (e.g., the percentage of non-diseased patients who are correctly characterized). The assay parameters can be adjusted to optimize for both sensitivity and specificity.

Table 1 lists miR that have elevated levels in serum from patients with EAC, elevated levels in EAC cell lines or both. Table 2 lists miR that have reduced levels in serum from patients with EAC, reduced levels in EAC cell lines or both. One or more of these miR may be used in accordance with the present invention. Some of the miR are useful for characterizing EAC, including distinguishing EAC from BE and/or distinguishing EAC from normal. Other miR are suitable markers for identifying patients with BE. In addition, some miR may be used to predict the aggressiveness or outcome of BE.

TABLE 1
MicroRNA Biomarkers Upregulated in EAC
MicroRNA ID (Sequence Identifier)

| | | |
|---|---|---|
| hsa-miR-30a* (SEQ ID NO: 1) | hsa-miR-206 (SEQ ID NO: 2) | hsa-miR-513_v9.1 (SEQ ID NO: 3) |
| hsa-miR-583 (SEQ ID NO: 4) | hsa-miR-629* (SEQ ID NO: | hsa-miR-515-3p_v9.1 (SEQ ID NO: 6) |
| hsa-miR-571 (SEQ ID NO: 7) | hsa-miR-33b_v9.1 (SEQ ID NO: 8) | hsa-miR-125b (SEQ ID NO: 9) |
| hsa-miR-522_v9.1 (SEQ ID NO: 10) | hsa-miR-199b-5p (SEQ ID NO: 11) | hsa-miR-191_v9.1 (SEQ ID NO: 12) |
| hsa-miR-9 (SEQ ID NO: 13) | hsa-miR-1_v9.1 (SEQ ID NO: 14) | hsa-miR-660 (SEQ ID NO: 15) |
| hsa-miR-765 (SEQ ID NO: 16) | hsa-miR-146b-5p (SEQ ID NO: 17) U | hsa-miR-135b_v9.1 (SEQ ID NO: 18) |
| hsa-miR-523_v9.1 (SEQ ID NO: 19) | hsa-miR-381 (SEQ ID NO: 20) | hsa-miR-490-3p (SEQ ID NO: 21) |
| hsa-miR-210 (SEQ ID NO: 22) | hsa-miR-200a (SEQ ID NO: 23) | hsa-miR-520b (SEQ ID NO: 24) |
| hsa-miR-147 (SEQ ID NO: 25) | Hsa-miR-16 (SEQ ID NO: 26) | hsa-miR-591 (SEQ ID NO: 27) |
| hsa-miR-668 (SEQ ID NO: 28) | hsa-miR-101_v9.1 (SEQ ID NO: 29) | hsa-miR-519b_v9.1 (SEQ ID NO: 30) |
| hsa-miR-128b_v9.1 (SEQ ID NO: 31) | hsa-miR-650 (SEQ ID NO: 32) | hsa-miR-518d-3p (SEQ ID NO: 33) |
| hsa-miR-423_v9.1 (SEQ ID NO: 34) | hsa-miR-19b (SEQ ID NO: 35) | hsa-miR-586 (SEQ ID NO: 36) |
| hsa-miR-639 (SEQ ID NO: 37) | hsa-miR-196a_v9.1 (SEQ ID NO: 38) | hsa-miR-187_v9.1 (SEQ ID NO: 39) |
| hsa-miR-520f_v9.1 (SEQ ID NO: 40) | hsa-miR-144_v9.1 (SEQ ID NO: 41) | hsa-miR-767-5p (SEQ ID NO: 42) |
| hsa-miR-592 (SEQ ID NO: 43) | hsa-miR-492 (SEQ ID NO: 44) | hsa-miR-95 (SEQ ID NO: 45) |
| hsa-miR-422b_v9.1 (SEQ ID NO: 46) | hsa-miR-526a_v9.1 (SEQ ID NO: 47) | hsa-miR-34b* (SEQ ID NO: 48) |
| hsa-miR-302b (SEQ ID NO: 49) | hsa-miR-602 (SEQ ID NO: 50) | hsa-miR-15b (SEQ ID NO: 51) |
| hsa-miR-30c (SEQ ID NO: 52) | hsa-miR-585 (SEQ ID NO: 53) | hsa-miR-493-3p_v9.1 (SEQ ID NO: 54) |
| hsa-miR-518a-3p (SEQ ID NO: 55) | hsa-miR-550* (SEQ ID NO: 56) | hsa-miR-483_v9.1 (SEQ ID NO: 57) |
| hsa-mIR-17-5p_v9.1 (SEQ ID NO: 58) | hsa-miR-500* (SEQ ID NO: 59) | hsa-miR-520e (SEQ ID NO: 60) |
| hsa-miR-520g (SEQ ID NO: 61) | hsa-miR-615_v9.1 (SEQ ID NO: 62) | hsa-miR-26b_v9.1 (SEQ ID NO: 63) |
| hsa-miR-449a (SEQ ID NO: 64) | hsa-miR-499_v9.1 (SEQ ID NO: 65) | hsa-miR-196b_v9.1 (SEQ ID NO: 66) |
| hsa-raiR-203 (SEQ ID NO: 67) | hsa-miR-548d-3p (SEQ ID NO: 68) | hsa-miR-548b-3p (SEQ ID NO: 69) |
| hsa-miR-126_v9.1 (SEQ ID NO: 70) | hsa-miR-518c_v9.1 (SEQ ID NO: 71) | hsa-miR-337_v9.1 (SEQ ID NO: 72) |
| hsa-miR-605 (SEQ ID NO: 73) | hsa-miR-551b (SEQ ID NO: 74) | hsa-miR-92_v9.1 (SEQ ID NO: 75) |
| hsa-miR-224_v9.1 (SEQ ID NO: 76) | hsa-miR-645 (SEQ ID NO: 77) | hsa-miR-190 (SEQ ID NO: 78) |
| hsa-miR-106a_v9.1 (SEQ ID NO: 79) | hsa-miR-517b (SEQ ID NO: 80) | hsa-miR-185_v9.1 (SEQ ID NO: 81) |
| hsa-let-7a (SEQ ID NO: 82) | hsa-miR-129-5p (SEQ ID NO: 83) | hsa-miR-299-3p (SEQ ID NO: 84) |
| hsa-miR-25 (SEQ ID NO: 85) | hsa-miR-604 (SEQ ID NO: 86) | hsa-miR-498 (SEQ ID NO: 87) |
| hsa-miR-449b (SEQ ID NO: 88) | hsa-miR-302a*_v9.1 (SEQ ID NO: 89) | hsa-miR-613 (SEQ ID NO: 90) |
| hsa-miR-519d_v9.1 (SEQ ID NO: 91) | hsa-miR-425 (SEQ ID NO: 92) | hsa-miR-328 (SEQ ID NO: 93) |
| hsa-miR-659 (SEQ ID NO: 94) | hsa-miR-380 (SEQ ID NO: 95) | hsa-miR-507 (SEQ ID NO: 96) |
| hsa-miR-302d (SEQ ID NO: 97) | hsa-miR-630 (SEQ ID NO: 98) | hsa-miR-433 (SEQ ID NO: 99) |
| hsa-miR-493* (SEQ ID NO: 100) | hsa-let-7f (SEQ ID NO: 101) | hsa-miR-184 (SEQ ID NO: 102) |
| hsa-miR-422a_v9.1 (SEQ ID NO: 103) | hsa-miR-587 (SEQ ID NO: 104) | hsa-miR-296-5p (SEQ ID NO: 105) |
| hsa-miR-573 (SEQ ID NO: 106) | hsa-miR-192 (SEQ ID NO: 107) | hsa-miR-556_v9.1 (SEQ ID NO: 108) |
| hsa-miR-374a (SEQ ID NO: 109) | hsa-miR-107 (SEQ ID NO: 110) | hsa-miR-659 (SEQ ID NO: 111) |
| hsa-miR-628_v9.1 (SEQ ID NO: 112) | hsa-miR-609 (SEQ ID NO: 113) | hsa-miR-18a*_v9.1 (SEQ ID NO: 114) |
| hsa-miR-485-3p (SEQ ID NO: 115) | hsa-miR-141 (SEQ ID NO: 116) | hsa-miR-519c-3p (SEQ ID NO: 117) |
| hsa-miR-132 (SEQ ID NO: 118) | hsa-miR-624* (SEQ ID NO: 119) | hsa-miR-20a (SEQ ID NO: 120) |
| hsa-miR-421 (SEQ ID NO: 121) | hsa-let-7e_v9.1 (SEQ ID NO: 122) | hsa-miR-212 (SEQ ID NO: 123) |
| hsa-miR-20b (SEQ ID NO: 124) | hsa-miR-576_v9.1 (SEQ ID NO: 125) | hsa-miR-526b_v9.1 (SEQ ID NO: 126) |
| hsa-miR-649 (SEQ ID NO: 127) | hsa-miR-519a_v9.1 (SEQ ID NO: 128) | hsa-miR-100 (SEQ ID NO: 129) |
| hsa-miR-607 (SEQ ID NO: 130) | hsa-miR-643 (SEQ ID NO: 131) | hsa-let-7g_v9.1 (SEQ ID NO: 132) |
| hsa-miR-98 (SEQ ID NO: 133) | hsa-miR-195 (SEQ ID NO: 134) | hsa-miR-603 (SEQ ID NO: 135) |
| hsa-miR-448 (SEQ ID NO: 136) | hsa-miR-34a_v9.1 (SEQ ID NO: 137) | hsa-miR-539 (SEQ ID NO: 138) |
| hsa-miR-520d*_v9.1 (SEQ ID NO: 139) | hsa-miR-568 (SEQ ID NO: 140) | hsa-miR-299-5p (SEQ ID NO: 141) |
| hsa-miR-34c-5p (SEQ ID NO: 142) | hsa-miR-152_v9.1 (SEQ ID NO: 143) | hsa-miR-553 (SEQ ID NO: 144) |
| hsa-miR-509_v9.1 (SEQ ID NO: 145) | hsa-miR-516a-3p (SEQ ID NO: 146) | hsa-miR-122a_v9.1 (SEQ ID NO: 147) |
| hsa-miR-644 (SEQ ID NO: 148) | hsa-miR-599 (SEQ ID NO: 149) | hsa-miR-514_v9.1 (SEQ ID NO: 150) |
| hsa-miR-512-3p (SEQ ID NO: 151) | hsa-miR-578 (SEQ ID NO: 152) | hsa-miR-561 (SEQ ID NO: 153) |
| hsa-miR-149_v9.1 (SEQ ID NO: 154) | hsa-miR-625_v9.1 (SEQ ID NO: 155) | hsa-miR-134_v9.1 (SEQ ID NO: 156) |
| hsa-imR-454-3p_v9.1 (SEQ ID NO: 157) | hsa-miR-373* (SEQ ID NO: 158) | hsa-miR-198_v9.1 (SEQ ID NO: 159) |
| hsa-miR-610 (SEQ ID NO: 160) | hsa-miR-658 (SEQ ID NO: 161) | hsa-miR-383 (SEQ ID NO: 162) |
| hsa-miR-30b (SEQ ID NO: 163) | hsa-miR-663 (SEQ ID NO: 164) | hsa-miR-552 (SEQ ID NO: 165) |
| hsa-miR-559e_v9.1 (SEQ ID NO: 166) | hsa-mriR-453_v9.1 (SEQ ID NO: 167) | hsa-miR-29b (SEQ ID NO: 168) |
| hsa-miR-127-3p (SEQ ID NO: 169) | hsa-miR-367 (SEQ ID NO: 170) | hsa-miR-545_v9.1 (SEQ ID NO: 171) |
| hsa-miR-532-5p (SEQ ID NO: 172) | hsa-miR-32_v9.1 (SEQ ID NO: 173) | hsa-miR-516b (SEQ ID NO: 174) |
| hsa-miR-200b_v9.1 (SEQ ID NO: 175) | hsa-miR-595 (SEQ ID NO: 176) | hsa-miR-362-5p (SEQ ID NO: 177) |
| hsa-miR-99a (SEQ ID NO: 178) | hsa-miR-601 (SEQ ID NO: 179) | hsa-miR-191* (SEQ ID NO: 180) |
| hsa-miR-128a_v9.1 (SEQ ID NO: 181) | hsa-miR-651 (SEQ ID NO: 182) | hsa-miR-590-5p (SEQ ID NO: 183) |
| hsa-miR-501-5p (SEQ ID NO: 184) | hsa-miR-526b* (SEQ ID NO: 185) | hsa-miR-487b (SEQ ID NO: 186) |
| hsa-miR-608 (SEQ ID NO: 187) | hsa-miR-508-3p (SEQ ID NO: 188) | hsa-miR-19a (SEQ ID NO: 189) |
| hsa-let-7i_v9.1 (SEQ ID NO: 190) | hsa-miR-518f* (SEQ ID NO: 191) | hsa-miR-181d_v9.1 (SEQ ID NO: 192) |
| hsa-miR-302c* (SEQ ID NO: 193) | hsa-miR-373 (SEQ ID NO: 194) | hsa-miR-517c (SEQ ID NO: 195) |
| hsa-let-7b (SEQ ID NO: 196) | hsa-miR-769-3p (SEQ ID NO: 197) | hsa-miR-655 (SEQ ID NO: 198) |
| hsa-miR-432 (SEQ ID NO: 199) | hsa-miR-325 (SEQ ID NO: 200) | hsa-miR-135a (SEQ ID NO: 201) |
| hsa-miR-7_v9.1 (SEQ ID NO: 202) | hsa-miR-193a_v9.1 (SEQ ID NO: 203) | hsa-miR-27b (SEQ ID NO: 204) |
| hsa-miR-202_v9.1 (SEQ ID NO: 205) | hsa-let-7d_v9.1 (SEQ ID NO: 206) | hsa-miR-562 (SEQ ID NO: 207) |
| hsa-miR-183_v9.1 (SEQ ID NO: 208) | hsa-miR-596 (SEQ ID NO: 209) | hsa-miR-622 (SEQ ID NO: 210) |
| hsa-miR-370_v9.1 (SEQ ID NO: 211) | hsa-miR-155_v9.1 (SEQ ID NO: 212) | hsa-miR-302c (SEQ ID NO: 213) |
| hsa-miR-524-3p (SEQ ID NO: 214) | hsa-miR-363 (SEQ ID NO: 215) | hsa-miR-582-5p (SEQ ID NO: 216) |
| hsa-miR-301a (SEQ ID NO: 217) | hsa-miR-138_v9.1 (SEQ ID NO: 218) | hsa-miR-520d_v9.1 (SEQ ID NO: 219) |
| hsa-miR-518f_v9.1 (SEQ ID NO: 220) | hsa-miR-28-5p (SEQ ID NO: 221) | hsa-let-7c (SEQ ID NO: 222) |
| hsa-miR-369-5p (SEQ ID NO: 223) | hsa-miR-450_v9.1 (SEQ ID NO: 224) | hsa-miR-137 (SEQ ID NO: 225) |
| hsa-miR-524-5p (SEQ ID NO: 226) | hsa-miR-520h (SEQ ID NO: 227) | hsa-miR-572 (SEQ ID NO: 228) |
| hsa-miR-495_v9.1 (SEQ ID NO: 229) | hsa-miR-758 (SEQ ID NO: 230) | hsa-miR-10b_v9.1 (SEQ ID NO: 231) |
| hsa-miR-580 (SEQ ID NO: 232) | hsa-miR-215 (SEQ ID NO: 233) | hsa-miR-378* (SEQ ID NO: 234) |
| hsa-miR-30e* (SEQ ID NO: 235) | hsa-miR-93 (SEQ ID NO: 236) | hsa-miR-521 (SEQ ID NO: 237) |
| hsa-miR-511 (SEQ ID NO: 238) | hsa-miR-379_v9.1 (SEQ ID NO: 239) | hsa-miR-431 (SEQ ID NO: 240) |
| hsa-miR-382 (SEQ ID NO: 241) | hsa-miR-632 (SEQ ID NO: 242) | hsa-miR-647 (SEQ ID NO: 243) |
| hsa-miR-182_v9.1 (SEQ ID NO: 244) | hsa-miR-154 (SEQ ID NO: 245) | hsa-miR-188_v9.1 (SEQ ID NO: 246) |
| hsa-miR-520a-3p (SEQ ID NO: 247) | hsa-miR-302a (SEQ ID NO: 248) | hsa-miR-451_v9.1 (SEQ ID NO: 249) |
| hsa-miR-133a_v9.1 (SEQ ID NO: 250) | hsa-miR-126* (SEQ ID NO: 251) | |

TABLE 2
MicroRNA Biomarkers Downregulated in EAC
MicroRNA ID (Sequence Identifier)

| | | |
|---|---|---|
| hsa-miR-24 (SEQ ID NO: 252) | hsa-miR-30d (SEQ ID NO: 253) | hsa-miR-221 (SEQ ID NO: 254) |
| hsa-miR-125a_v9.1 (SEQ ID NO: 255) | hsa-miR-335 (SEQ ID NO: 256) | hsa-miR-342_v9.1 (SEQ ID NO: 257) |
| hsa-miR-324-5p (SEQ ID NO: 258) | hsa-miR-18a_v9.1 (SEQ ID NO: 259) | hsa-miR-181a (SEQ ID NO: 260) |
| hsa-miR-575 (SEQ ID NO: 261) | hsa-miR-365 (SEQ ID NO: 262) | hsa-miR-27a (SEQ ID NO: 263) |
| hsa-miR-l30b (SEQ ID NO: 264) | hsa-miR-193b_v9.1 (SEQ ID NO: 265) | hsa-miR-222_v9.1 (SEQ ID NO: 266) |
| hsa-miR-200c_v9.1 (SEQ ID NO: 267) | hsa-miR-106b (SEQ ID NO: 268) | hsa-miR-361-5p (SEQ ID NO: 269) |
| hsa-miR-326 (SEQ ID NO: 270) | hsa-miR-148b (SEQ ID NO: 271) | hsa-miR-151-3p (SEQ ID NO: 272) |
| hsa-miR-410 (SEQ ID NO: 273) | hsa-miR-527_v9.1 (SEQ ID NO: 274) | hsa-miR-205 (SEQ ID NO: 275) |
| hsa-miR-99b (SEQ ID NO: 276) | hsa-miR-15a (SEQ ID NO: 277) | hsa-miR-23a (SEQ ID NO: 278) |
| hsa-miR-636_v9.1 (SEQ ID NO: 279) | hsa-miR-638 (SEQ ID NO: 280) | hsa-miR-345_v9.1 (SEQ ID NO: 281) |
| hsa-miR-519e* (SEQ ID NO: 282) | hsa-miR-654-5p (SEQ ID NO: 283) | hsa-miR-142-3p (SEQ ID NO: 284) |
| hsa-miR-31_v9.1 (SEQ ID NO: 285) | hsa-miR-133b (SEQ ID NO: 286) | hsa-miR-766 (SEQ ID NO: 287) |
| hsa-miR-770-5p (SEQ ID NO: 288) | hsa-miR-136 (SEQ ID NO: 289) | hsa-miR-640 (SEQ ID NO: 290) |
| hsa-miR-30e-5p_v9.1 (SEQ ID NO: 291) | hsa-miR-30a (SEQ ID NO: 292) | hsa-miR-409-3p (SEQ ID NO: 293) |
| hsa-miR-17-3p_v9.1 (SEQ ID NO: 294) | hsa-miR-29a_v9.1 (SEQ ID NO: 295) | hsa-miR-130a (SEQ ID NO: 296) |
| hsa-miR-194 (SEQ ID NO: 297) | hsa-miR-338_v9.1 (SEQ ID NO: 298) | hsa-miR-525-5p (SEQ ID NO: 299) |
| hsa-miR-92b_v9.1 (SEQ ID NO: 300) | hsa-miR-23b (SEQ ID NO: 301) | hsa-miR-652_v9.1 (SEQ ID NO: 302) |
| hsa-miR-491_v9.1 (SEQ ID NO: 303) | hsa-miR-623 (SEQ ID NO: 304) | hsa-miR-331-3p (SEQ ID NO: 305) |
| hsa-miR-346 (SEQ ID NO: 306) | hsa-miR-574_v9.1 (SEQ ID NO: 307) | hsa-miR-614 (SEQ ID NO: 308) |
| hsa-miR-214_v9.1 (SEQ ID NO: 309) | hsa-miR-145_v9.1 (SEQ ID NO: 310) | hsa-miR-648 (SEQ ID NO: 311) |
| hsa-miR-371-3p (SEQ ID NO: 312) | hsa-miR-22 (SEQ ID NO: 313) | hsa-miR-199a-5p (SEQ ID NO: 314) |
| hsa-miR-504_v9.1 (SED ID NO: 315) | hsa-miR-564 (SEQ ID NO: 316) | hsa-miR-497 (SEQ ID NO: 317) |
| hsa-miR-557 (SEQ ID NO: 318) | hsa-miR-320_v9.1 (SEQ ID NO: 319) | hsa-miR-21 (SEQ ID NO: 320) |
| hsa-miR-486-5p (SEQ ID NO: 321) | hsa-miR-146a (SEQ ID NO: 322) | hsa-miR-620 (SEQ ID NO: 323) |
| hsa-miR-186_v9.1 (SEQ ID NO: 324) | hsa-miR-375 (SEQ ID NO: 325) | hsa-miR-671_v9.1 (SEQ ID NO: 326) |
| hsa-miR-617 (SEQ ID NO: 327) | hsa-miR-376a (SEQ ID NO: 328) | hsa-miR-542-5p_v9.1 (SEQ ID NO: 329) |
| hsa-miR-143_v9.1 (SEQ ID NO: 330) | hsa-miR-662 (SEQ ID NO: 331) | hsa-miR-223_v9.1 (SEQ ID NO: 332) |
| hsa-miR-330-3p (SEQ ID NO: 333) | hsa-miR-33_v9.1 (SEQ ID NO: 334) | hsa-miR-502-5p (SEQ ID NO: 335) |
| hsa-miR-339_v9.1 (SEQ ID NO: 336) | hsa-miR-627 (SEQ ID NO: 337) | hsa-miR-197 (SEQ ID NO: 338) |
| hsa-miR-29c_v9.1 (SEQ ID NO: 339) | hsa-miR-584 (SEQ ID NO: 340) | hsa-miR-199a*_v9.1 (SEQ ID NO: 341) |
| hsa-miR-452*_v9.1 (SEQ ID NO: 342) | hsa-miR-181c (SEQ ID NO: 343) | hsa-miR-142-5p_v9.1 (SEQ ID NO: 344) |
| hsa-miR-148a (SEQ ID NO: 345) | hsa-miR-631 (SEQ ID NO: 346) | hsa-miR-517* (SEQ ID NO: 347) |
| hsa-miR-452_v9.1 (SEQ ID NO: 348) | hsa-miR-518c* (SEQ ID NO: 349) | hsa-miR-566 (SEQ ID NO: 350) |
| hsa-miR-484 (SEQ ID NO: 351) | hsa-miR-103 (SEQ ID NO: 352) | hsa-miR-424 (SEQ ID NO: 353) |
| hsa-miR-510_v9.1 (SEQ ID NO: 354) | hsa-miR-769-5p (SEQ ID NO: 355) | hsa-miR-140-5p (SEQ ID NO: 356) |
| hsa-miR-494_v9.1 (SEQ ID NO: 357) | hsa-miR-377 (SEQ ID NO: 358) | hsa-miR-18b_v9.1 (SEQ ID NO: 359) |
| hsa-miR-425-3p_v9.1 (SEQ ID NO: 360) | hsa-miR-324-3p (SEQ ID NO: 361) | hsa-miR-10a (SEQ ID NO: 362) |
| hsa-miR-26a_v9.1 (SEQ ID NO: 363) | hsa-miR-139_v9.1 (SEQ ID NO: 364) | hsa-miR-181b_v9.1 (SEQ ID NO: 365) |

### III. Samples Containing MicroRNA

The terms “sample,” “biological sample,” “patient sample” and the like, encompass a variety of sample types obtained from an individual, subject or a patient and can be used in a diagnostic or monitoring assay. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, serum, plasma, urine, saliva, stool and synovial fluid), solid tissue samples such as a biopsy specimen or tissue cultures or cells derived therefrom and the progeny thereof. The definition also includes samples that have been manipulated in any way after their procurement, such as by centrifugation, filtration, precipitation, dialysis, chromatography, treatment with reagents, washed or enriched for certain cell populations including tumor cells and the like. The terms further encompass a clinical sample, and also include cells in culture, cell supernatants, tissue samples, organs, bone marrow, and the like. In a specific embodiment, a sample comprises a blood sample.

In another embodiment, a serum sample is used. Serum is typically the fluid, non-cellular portion of coagulated blood. Plasma is also a non-cellular blood sample, but unlike serum, plasma contains clotting factors. In some embodiments, serum or plasma samples may be obtained from a human patient previously screened for EAC using known diagnostic methods. In other embodiments, the patient has undergone a physical exam, endoscopy, esophagogastroduodenoscopy or biopsy to detect EAC. Additional embodiments include measuring miR in samples from patients previously or currently undergoing treatment for EAC or BE. The volume of plasma or serum obtained and used for the assay may be varied depending upon clinical intent.

Methods for obtaining and preparing serum samples are known in the art. Generally, blood is drawn into a collection tube using standard methods and allowed to clot. The serum is then separated from the cellular portion of the coagulated blood. In some methods, clotting activators such as silica particles are added to the blood collection tube. In other methods, the blood is not treated to facilitate clotting. Blood collection tubes are commercially available from many sources and in a variety of formats (e.g., Becton Dickinson Vacutainer® SST, glass serum tubes, or plastic serum tubes).

In some methods, the blood is collected by venipuncture and processed within three hours after drawing to minimize hemolysis and minimize the release of miR from intact cells in the blood. In some methods, blood is kept on ice until use. The blood may be fractionated by centrifugation to remove cellular components. In some embodiments, centrifugation to prepare serum can be at a speed of at least about 500, at least about 1000, at least about 2000, at least about 3000, at least about 4000, or at least about 5000×G. In certain embodiments, the blood can be incubated for at least about 10, at least about 20, at least about 30, at least about 40, at least about 50, at least about 60, at least about 90, at least about 120, or at least about 150 minutes to allow clotting. In other embodiments, the blood is incubated for at most 3 hours. When using plasma, the blood is not permitted to coagulate prior to separation of the cellular and acellular components. Serum or plasma can be frozen after separation from the cellular portion of blood until further assayed.

Before analysis, RNA may be extracted from serum or plasma and purified using methods known in the art. Many methods are known for isolating total RNA, or to specifically extract small RNAs, including miR. The RNA may be extracted using commercially-available kits (e.g., miRNeasy Mini Kit (QIAGEN, Inc. (Valencia, Calif.)); Perfect RNA Total RNA Isolation Kit (5 Prime, Inc. (Gaithersburg, Md.)); and mirVana™ miRNA Isolation Kit (Ambion, Inc. (Austin, Tex.))). Alternatively, RNA extraction methods previously published for the extraction of mammalian intracellular RNA or viral RNA may be adapted, either as published or with modification, for extraction of RNA from plasma and serum. RNA may be extracted from plasma or serum using silica particles, glass beads, or diatoms, as in the method or adaptations described in U.S. Patent Application Publication No. 2008/0057502.

### IV. Methods to Measure the Level of MicroRNA

Many methods of measuring the levels or amounts of miR are contemplated. Any reliable, sensitive, and specific method can be used. In particular embodiments, a miR is amplified prior to measurement. In other embodiments, the level of miR is measured during the amplification process. In still other methods, the miR is not amplified prior to measurement.

A. Amplification Reactions

Many methods exist for amplifying miR nucleic acid sequences such as mature miR, precursor miR, and primary miR. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-medicated amplification (TMA), RNA (Eberwine) amplification, and other methods that are known to person skilled in the art. In certain embodiments, more than one amplification method is used, such as reverse transcription followed by real time quantitative PCR (qRT-PCR). See, e.g., Chen et al., 33(20) NUCL. ACIDS RES. e179 (2005).

A typical PCR reaction comprises multiple amplifications steps or cycles that selectively amplify target nucleic acid species including a denaturing step in which a target selectively amplify target nucleic acid species including a denaturing step in which a target nucleic acid is denatured; an annealing step in which a set of PCR primers (forward and reverse primers) anneal to complementary DNA strands; and an extension step in which a thermostable DNA polymerase extends the primers. By repeating these steps multiple times, a DNA fragment is amplified to produce an amplicon, corresponding to the target DNA sequence. Typical PCR reactions include about 20 or more cycles of denaturation, annealing, and extension. In many cases, the annealing and extension steps can be performed concurrently, in which case the cycle contains only two steps. Because mature miR are single-stranded, a reverse transcription reaction (which produces a complementary cDNA sequence) may be performed prior to PCR reaction. Reverse transcription reactions include the use of, e.g., a RNA-based DNA polymerase (reverse transcriptase) and a primer.

In PCR and q-PCR methods, for example, a set of primers is used for each target sequence. In certain embodiments, the lengths of the primers depends on many factors, including, but not limited to, the desired hybridization temperature between the primers, the target nucleic acid sequence, and the complexity of the different target nucleic acid sequences to be amplified. In certain embodiments, a primer is about 15 to about 35 nucleotides in length. In other embodiments, a primer is equal to or fewer than about 15, fewer than about 20, fewer than about 25, fewer than about 30, or fewer than about 35 nucleotides in length. In additional embodiments, a primer is at least about 35 nucleotides in length.

In a further embodiment, a forward primer can comprise at least one sequence that anneals to a miR biomarker and alternatively can comprise an additional 5′ non-complementary region. In another embodiment, a reverse primer can be designed to anneal to the complement of a reverse transcribed miR. The reverse primer may be independent of the miR biomarker sequence, and multiple miR biomarkers may be amplified using the same reverse primer. Alternatively, two or more miR are amplified in a single reaction volume. One aspect includes multiplex q-PCR, such as qRT-PCR, which enables simultaneous amplification and quantification of at least two miR of interest in one reaction volume by using more than one pair of primers and/or more than one probe. The primer pairs comprise at least one amplification primer that uniquely binds each miR, and the probes are labeled such that they are distinguishable from one another, thus allowing simultaneous quantification of multiple miR. Multiplex qRT-PCR has research and diagnostic uses including, but not limited, to detection of miR for diagnostic, prognostic, and therapeutic applications.

The qRt-PCR reaction may further be combined with the reverse transcription reaction by including both a reverse transcriptase and a DNA-based thermostable DNA polymerase. When two polymerases are used, a “hot start” approach may be used to maximize assay performance. See U.S. Pat. No. 5,985,619 and U.S. Pat. No. 5,411,876. For example, the components for a reverse transcriptase reaction and a PCR reaction may be sequestered using one or more thermoactivation methods or chemical alteration to improve polymerization efficiency. See U.S. Pat. No. 6,403,341; U.S. Pat. No. 5,550,044; and U.S. Pat. No. 5,413,924.

B. Detection of MicroRNA

In certain embodiments, labels, dyes, or labeled probes and/or primers are used to detect amplified or unamplified miR. One of ordinary skill in the art will recognize which detection methods are appropriate based on the sensitivity of the detection method and the abundance of the target. Depending on the sensitivity of the detection method and the abundance of the target, amplification may or may not be required prior to detection. One skilled in the art will recognize the detection methods where miR amplification is preferred.

A probe or primer may include Watson-Crick Bases or modified bases. Modified bases include, but are not limited to, the AEGIS bases (from EraGen Biosciences, Inc. (Madison, Wis.)), which have been described, e.g., in U.S. Pat. No. 6,001,983; U.S. Pat. No. 5,965,364; and U.S. Pat. No. 5,432,272. In certain aspects, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809.

In a further aspect, oligonucleotide probes or primers present in an amplification reaction are suitable for monitoring the amount of amplification product produced as a function of time. In certain aspects, probes having different single stranded versus double stranded character are used to detect the nucleic acid. Probes include, but are not limited to, the 5′-exonuclease assay (e.g., TaqMan®) probes (see U.S. Pat. No. 5,538,848), stem-loop molecular beacons (see, e.g., U.S. Pat. No. 6,103,476 and U.S. Pat. No. 5,925,517), stemless or linear becaons (see, e.g., WO 9921881, U.S. Pat. No. 6,649,349 and U.S. Pat. No. 6,485,901), peptide nucleic acid (PNA) Molecular Beacons (see, e.g., U.S. Pat. No. 6,593,091 and U.S. Pat. No. 6,355,421), linear PNA beacons (see, e.g., U.S. Pat. No. 6,329,144), non-FRET probes (see, e.g., U.S. Pat. No. 6,150,097), Sunrise®/Amplifluor® probes (see, e.g., U.S. Pat. No. 6,548,250), stem-loop and duplex Scorpion™ probes (see, e.g., U.S. Pat. No. 6,589,743), bulge loop probes (see, e.g., U.S. Pat. No. 6,590,091), pseudo knot probes (see, e.g., U.S. Pat. NO. 6,548,250), cyclicons (see, e.g., U.S. Pat. No. 6,383,752), MGB Eclipse® probe (Sigma-Aldrich Corp. (St. Louis, Mo.)), hairpin probes (see, e.g., U.S. Pat. No. 6,596,490), PNA light-up probes, antiprimer quench probes (Li et al., 53 CLIN. CHEM. 624-33 (2006)), self-assembled nanoparticle probes, and ferrocene-modified probes described, for example, in U.S. Pat. No. 6,485,901.

In certain embodiments, one or more of the primers in an amplification reaction can include a label. In yet further embodiments, different probes or primers comprise detectable labels that are distinguishable from one another. In some embodiments a nucleic acid, such as the probe or primer, may be labeled with two or more distinguishable labels.

In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other aspects, use of labels can be accomplished using any one of a large number of known techniques employing known labels, linkages, linking groups, reagents, reaction conditions, and analysis and purification methods.

MicroRNA can be detected by direct or indirect methods. In a direct detection method, one or more miR are detected by a detectable label that is linked to a nucleic acid molecule. In such methods, the miR may be labeled prior to binding to the probe. Therefore, binding is detected by screening for the labeled miR that is bound to the probe. The probe is optionally linked to a bead in the reaction volume.

In certain embodiments, nucleic acids are detected by direct binding with a labeled probe, and the probe is subsequently detected. In one embodiment of the invention, the nucleic acids, such as amplified miR, are detected using xMAP Microspheres (Luminex Corp. (Austin, Tex.)) conjugated with probes to capture the desired nucleic acids. Some methods may involve detection with polynucleotide probes modified, for example, with fluorescent labels or branched DNA (bDNA) detection.

In other embodiments, nucleic acids are detected by indirect detection methods. For example, a biotinylated probe may be combined with a stretavidin-conjugated dye to detect the bound nucleic acid. The streptavidin molecule binds a biotin label on amplified miR, and the bound miR is detected by detecting the dye molecule attached to the streptavidin molecule. In one embodiment, the streptavidin-conjugated dye molecule comprises Phycolink® Streptavidin R-Phycoerythrin (ProZyme, Inc. (Heard, Calif.)). Other conjugated dye molecules are known to persons skilled in the art.

Labels include, but are not limited to, light-emitting, light-scattering, and light-absorbing compounds which generate or quench a detectable fluorescent, chemiluminescent, or bioluminescent signal. See, e.g., Garman A., Non-Radioactive Labeling, Academic Press (1997) and Kricka, L., Nonistopic DNA Probe Techniques, Academic Press, San Diego (1992). Fluorescent reporter dyes useful as labels include, but are not limited to, fluoresceins (see, e.g., U.S. Pat. No. 6,020,481; U.S. Pat. No. 6,008,379; and U.S. Pat. No. 5,188,934), rhodamines (see, e.g., U.S. Pat. No. 6,191,278; U.S. Pat. No. 6,051,719; U.S. Pat. No. 5,936,087; U.S. Pat. No. 5,847,162; and U.S. Pat. No. 5,366,860), benzophenoxazines (see, e.g., U.S. Pat. No. 6,140,500), energy-transfer fluorescent dyes, comprising pairs of donors and acceptors (see, e.g., U.S. Pat. No. 5,945,526; U.S. Pat. No. 5,863,727; and U.S. Pat. No. 5,800,996; and), and cyanines (see, e.g., WO 9745539), lissamine, phycoerythrin, Cy2, Cy3, Cy3.5, Cy5, Cy5.5, Cy7, FluorX (Amersham Biosciences, Inc. (Piscataway, N.J.)), Alexa 350, Alexa 430, AMCA, BODIPY 620/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, Hex, 6-JOE, Oregon Green 488, Oregon Green 500, Oregon Green 514, Pacific Blue, REG, Rhodamine Green, Rhodamine Red, Renographin, ROX, SYPRO, TAMRA, Tetramethylrhodamine, and/or Texas Red, as well as any other fluorescent moiety capable of generating a detectable signal. Examples of fluorescein dyes include, but are not limited to, 6-carboxyfluorescein, 2,′,4′,1,4-tetrachlorofluorescein, and 2′,4′,5′,7′,1,4-hexachlorofluorescein. In certain aspects, the fluorescent label is selected from SYBR-Green, 6-carboxyfluorescein (“FAM”), TET, ROX, VICTM, and JOE. For example, in certain embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054. A dual labeled fluorescent probe that includes a reporter fluorophore and a quencher fluorophore is used in some embodiments. It will be appreciated that pairs of fluorophores are chosen that have distinct emission spectra so that they can be easily distinguished.

In further embodiments, labels are hybridization-stabilizing moieties which serve to enhance, stabilize, or influence hybridization of duplexes, e.g., interealators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In further aspects, methods relying on hybridization and/or ligation to quantify miR may be used including, but not limited to, oligonucleotide ligation (OLA) methods and methods that allow a distinguishable probe that hybridizes to the target nucleic acid sequence to be separated from an unbound probe. For example, HARP-like probes, as disclosed in U.S. Parent Application Publication No. 2006/0078894 may be used to measure the quantity of miR. In such methods, after hybridization between a probe and the targeted nucleic acid, the probe is modified to distinguish the hybridized probe from the unhybridized probe. Thereafter, the probe may be amplified and/or detected. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify the unhybridized HARP probe such that it cannot be amplified.

In an additional embodiment of the method, a probe ligation reaction may be used to quantify miR. In a Multiplex Ligation-dependent Probe Amplification (MLPA) technique, pairs of probes which hybridize immediately adjacent to each other on the target nucleic acid are ligated to each other only in the presence of the target nucleic acid. See Schouten et al., 30 NUCL. ACIDS RES. e57 (2002). In some aspects, MLPA probes have flanking PCR primer binding sites. MLPA probes can only be amplified if they have been ligated, thus allowing for detection and quantification of miR biomarkers.

Furthermore, a sample may also be analyzed by means of a microarray. Microarrays generally comprise solid substrates and have a generally planar surface, to which a capture reagent (also called an adsorbent or affinity reagent) is attached. Frequently, the surface of a microarray comprises a plurality of addressable locations, each of which has the capture reagent (e.g., miR probes specific for particular biomarkers) bound there. Many microarrays are described in the art. These include, for example, miR biochips produced by Asuragen, Inc. (Austin, Tex.); Affymetric, Inc. (Santa Clara, Calif.); GenoSensor Corp. (Tempe, Ariz.); Invitrogen, Corp. (Carlsbad, Calif.); and Illumina, Inc. (San Diego, Calif.).

### V. Determination of Characterization of Cancer Status

The present invention relates to the use of biomarkers to detect cancer. More specifically, the biomarkers of the present invention can be used in diagnostic tests to determine, characterize, qualify, and/or assess cancer status, for example, to diagnose cancer, in an individual, subject or patient. In particular embodiments, the cancer is EAC.

A. MicroRNA Biomarker Panels

The miR biomarkers of the present invention can be used in diagnostic tests to assess, characterize, determine, and/or qualify (used interchangeable herein) cancer status in a subject. The phrase “cancer status” includes any distinguishable manifestation of the disease, including non-disease. For example, cancer status includes, without limitation, the presence or absence of cancer (e.g., distinguishing between unaffected individuals (UI) and EAC in a subject), the risk of developing cancer (e.g., distinguishing between UI and BE in a subject or distinguishing between BE and EAC in a subject), the stage of the cancer, the progress of cancer (e.g., progress of cancer or remission of cancer over time) and the effectiveness or response to treatment of cancer (e.g., clinical follow up and surveillance of BE and EAC after treatment). Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the miR biomarker panels of the present invention may show a statistical difference in different cancer statuses of at least about p<0.05, at least about p<10⁻², at least about p<10⁻³, at least about p<10⁻⁴ or at least about p<10⁻⁵. Diagnostic tests that use these miR biomarkers may show a sensitivity and specificity of at least about 70%, at least about 75%, at least about 80%, at least about 85%, at least about 90%, at least about 95%, at least about 98% and about 100%.

The miR biomarkers are differentially present in UI (or normal control individuals (NC)), BE, and EAC, and, therefore, are useful in aiding in the determination of cancer status. IN certain embodiments, the miR biomarkers are measured in a patient sample using the methods described herein. The measurement(s) may then be compared with a relevant disgnostic amount(s) or cut-off(s) that distinguish a positive caner status from a negative cancer status. The diagnostic amount(s) represents a measured amount of a miR biomarker(s) above which or below which a subject is classified as having a particular cancer status. For example, if the miR biomarker(s) is/are up-regulated compared to normal during cancer (e.g., elevated levels), then a measured amount(s) above the diagnostic cutoff(s) provides a diagnosis of cancer. Alternatively, if the biomarker(s) is/are down-regulated during cancer (e.g., reduced levels), then a measured amount(s) below the diagnostic cuttoff(s) provides a diagnosis of cancer. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of the miR biomarker(s) in a statistically significant number of samples from subjects with the different cancer statuses, and drawing the cut-off to suit the desired levels of specificity and sensitivity.

Indeed, as the skilled artisan will appreciate there are many ways to use the measurements of two or more miR biomarkers in order to improve the diagnostic question under investigation. In a quite simple, but nonetheless often effective approach, a positive result is assumed if a sample is positive for at least one of the markers investigated.

Frequently, however, the combination of miR biomarkers is evaluated. In particular embodiments, the values measured for biomarkers of a miR biomarker panel are mathematically combined and the combined value is correlated to the underlying diagnostic question. MicroRNA biomarker values may be combined by any appropriate state of the art mathematical method. Well-known mathematical methods for correlating a marker combination to a disease employ methods like discriminant analysis (DA) (e.g., linear-, quadratic-, regularized-DA), Kernel Methods (e.g., SVM), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will recognize an appropriate method to evaluate a miR biomarker combination of the present invention. In certain embodiments, the method used in correlating miR biomarker combination of the present invention is selected from DA (e.g., Linear-, Quadratic- and Regularized DA), Kernel Methods (e.g., SVM), Nonparametric Methods (e.g., k-Nearest-Neighbor Classifiers), PLS (Partial Least Squares), Tree-Based Methods (e.g., Logic Regression, CART, random Forest Methods, Boosting Methods), or Generalized Linear Models (e.g., Logistic Regression). Details relating to these statistical methods are found in the following references: Pepe, M. S., The Statistical Evaluation of Medical Tests for Classification and Prediction, Oxford Statistical Science Series, 28 (2003); Ruczinski et al., 12 J. OF COMPUTATIONAL AND GRAPHICAL STATISTICS 475-511 (2003); Breiman, L., 45 MACHINE LEARNING 5-32 (2001); Duda, R. O., Hart, P. E., Stork, D. G., Pattern Classification, Wiley Interscience, 2nd Edition (2001); Hastie, Trevor, Tibshirani, Robert, and Friedman, Jerome, The Elements of Statistical Learning, Springer Series in Statistics (2001); Friedman, J. H., 84 J. OF THE AMERICAN STATISTICAL ASSOCIATION 165-75 (1989); Breiman, L., Friedman, J. H., Olshen, R. A., and Stone, C. J. Classification and regression trees, California: Wadsworth (1984).

B. Determining Risk of Developing Cancer

In a specific embodiment, the present invention provides methods for determining the risk of developing cancer in a subject. MicroRNA biomarker amounts of patterns are characteristic of various risk states, e.g., high, medium or low. The risk of developing a cancer is determined by measuring the relevant miR biomarkers and then either submitting them to a classification algorithm or comparing them with a reference amount and/or pattern of miR biomarkers that is associated with the particular risk level.

C. Determining Stage of Cancer

In another embodiment, the present invention provides methods for determining the stage of cancer in a subject. Each stage of the cancer has a characteristic amount of a miR biomarker or relative amounts of a set of miR biomarkers (a pattern). The stage of a cancer is determined by measuring the relevant miR biomarkers and then either submitting them to a classification algorithm or comparing them with a reference amount and/or pattern of miR biomarkers that is associated with the particular stage.

D. Determining Course (Progression/Remission) of Cancer

In one embodiment, the present invention provides methods for determining the course of cancer in a subject. Cancer course refers to changes in cancer status over time, including cancer progression (worsening) and cancer regression (improvement). Over time, the amounts of relative amounts (e.g., the pattern) of the miR biomarkers change. For example, miR biomarker “X” is increased with EAC, while biomarker “Y” may be decreased in EAC. Therefore, the trend of these miR biomarkers, either increased or decreased over time toward cancer or non-cancer indicates the course of the disease. Accordingly, this method involved measuring one or more miR biomarkers in a subject at least two different time points, e.g., a first time and a second time, and comparing the change in amounts, if any. The course of cancer is determined based on these comparisons.

E. Subject Management

In certain embodiments of the methods of qualifying cancer status, the methods further comprise managing subject treatment based on the status. Such management includes the actions of the physician or clinician subsequent to determining cancer status. For example, if a physician makes a diagnosis of BE, then a certain regime of monitoring (i.e., periodic endoscopy) would follow. A diagnosis of EAC may then require a certain cancer therapy regimen. Alternatively, a diagnosis of non-EAC might be followed with further testing to determine a specific disease that the patient might be suffering from. Also, further tests may be called for if the diagnostic test gives an inconclusive result on cancer status.

F. Determining Therapeutic Efficacy of Pharmaceutical Drug

In another embodiment, the present invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of one or more of the miR biomarkers of the present invention may change toward a non-cancer profile. Therefore, one can follow the course of the amounts of one or more biomarkers in the subject during the course of treatment. Accordingly, this method involves measuring one or more miR biomarkers in a subject receiving drug therapy, and correlating the amounts of the miR biomarkers with the cancer status of the subject. One embodiment of this method involves determining the levels of one or more miR biomarkers at least two different time points during a course of drug therapy, e.g., a first time and a second time, and comparing the change in amounts of the miR biomarkers, if any. For example, the one or more miR biomarkers can be measured before and after drug administration or at two different time points during drug administration. The effect of therapy is determined based on these comparisons. If a treatment is effective, then one or more miR biomarkers will trend toward normal, while if treatment is ineffective, the one or more miR biomarkers will trend toward cancer indications.

G. Generation of Classification Algorithms for Qualifying Cancer Status

In some embodiments, data that are generated using samples such as “known samples” can then be used to “train” a classification model. A “known sample” is a sample that has been pre-classified. The data that are used to form the classification model can be referred to as a “training data set.” The training data set that is used to form the classification model may comprise raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain et al., “Statistical Pattern recognition: A Review”, 22(1) IEEE TRANSACTIONS ON PATTERN ANALYSIS AND MACHINE INTELLIGENC (2000).

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning process such as CART-classification and regression trees), artificial neural networks such as back propagation networks, discriminant analyses (e.g., Bayesian classifier or Fischer analysis), logistic classifiers, and support vector classifiers (support vector machines). Another supervised classification method is a recursive partitioning process. Recursive partitioning processes use recursive partitioning trees to classify data derived from unknown samples. See U.S. Patent Application Publication No. 2002/0138208.

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm. Learning algorithms asserted for use in classifying biological information are described, for example, in WO 01/31580, U.S. Patent Applications Publication No. 2003/005615, No. 2003/0004402, and No. 2002/0193950.

The classification models can be formed on and used on any suitable digital computer. Suitable digital computers include micro, mini, or large computers using any standard or specialized operating system, such as a Unix, Window® or Linux™ based operating system. The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the miR biomarkers already discovered, or for finding new miR biomarkers. The classification algorithms, in turn, form the base for diagnostic tests by providing diagnostic values (e.g., cut-off points) for miR biomarkers used singly or in combination.

H. Kits for the Detection of Cancer Biomarkers

In another aspect, the present invention provides kits for qualifying cancer status, which kits are used to detect the miR biomarkers described herein. In a specific embodiment, the kit provided is a polymerase chain reaction kit comprising primers to the microRNA biomarkers of the present invention including, but not limited to, hsa-miR-345, hsa-miR-373*, hsa-miR-630, hsa-miR-765, hsa-miR-625, hsa-miR-93, hsa-miR-106b, hsa-miR-155, hsa-miR-130b, hsa-miR-30a, hsa-miR-301, and hsa-miR-15b.

The kit may further comprise a solid support, such as a chip, microtiter plate (e.g., a 96-well plate), microfuge tubes, and the like. The kit may further comprise a means for detecting the biomarkers.

A cancer patient can be diagnosed by adding blood or blood serum from the patient to the kit and detecting the relevant miR biomarkers, specifically, by a method which comprises the steps of: (i) collecting blood or blood serum from the patient; (ii) separating blood serum from the patient's blood; (iii) adding the blood serum from patient to a diagnostic kit; (iv) amplifying the miR biomarkers with appropriate primers; and, (v) detecting and/or measuring the amplified miR biomarkers. In this method, the primers are brought into contact with the patient's blood. If the miR biomarkers are present in the sample, the primers will bind to the sample, or a portion thereof, and amplification will occur. In other kit and diagnostic embodiments, blood or blood serum need not be collected from the patient (i.e., it is already collected).

The kit can also comprise appropriate solutions to maximize amplification conditions and/or instructions for making such solutions. In a further embodiment, a kit can comprise instructions for suitable operational parameters in the form of a label or separate insert. For example, the instructions may inform a consumer about how to collect the sample, how to perform the amplification and detection steps, etc. In yet another embodiment, the kit can comprise one or more containers with biomarker samples, to be used as standard(s) for calibration, as well as applicable controls.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Generation of MicroRNA Biomarker Panel for EAC

MiR microarrays were hybridized to miRs extracted from matching tissues and blood obtained from 16 subjects each with esophageal adenocarcinoma (EAC), and compared to that of 12 healthy subjects. In addition to these samples, miRs extracted from various normal esophageal, Barrett's, and EAC cell lines (HEEPiC, CHTRT, GiHTRT, QHTRT, and OE33) were also used. For these experiments, QIAGEN's miRNeasy Mini Kit was used for the actual miR extraction. Agilent's Human miR Microarray V1, which contains 471 human miRs, was used for hybridization. MiR-array data generated was normalized either by Agilent's GeneSpring GX 11.5 software or by the array control small RNA called Hurs. The normalized data was analyzed using significance analysis of microarrays (SAM). From the serum data, the data was first normalized the data using Hurs. The top 144 highest fold-change upregulated miRs that differ by a significant p-value between diseased and normal control (NC) were selected (Table 3). As a final filtering criterion, to ensure that serum miRs would be robustly detectable, miRs whose individual serum levels uniformly exceed array background by at least a factor of 5 were chosen. Next, the same data was normalized using GeneSpring GX 11.5, which used percentile shift normalization. This method generated 7 possible miR candidates. Then, the cell line data which was processed in the same way as the serum data was examined. The cell line data SAM result generated 11 possible miR candidates. Fourteen miR candidates were selected for further validation (hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, hsa-miR-15b) which commonly appeared or were deemed significant in 3 separate analyses.

TABLE 3
List of 144 Candidate MicroRNA Biomarkers
for EAC Using Hur-Based Normalization

| Gene ID | q-value (%) | Fold Change | P-value |
|---|---|---|---|
| hsa-miR-519e* | 0 | 9.36 | 0.0004 |
| hsa-miR-527_v9.1 | 0 | 6.84 | 0.0006 |
| hsa-miR-671_v9.1 | 0 | 4.97 | 0.0007 |
| hsa-miR-638 | 0 | 5.04 | 0.0008 |
| hsa-miR-520a-3p | 0 | 12.43 | 0.0009 |
| hsa-miR-181a* | 0 | 5.99 | 0.0014 |
| hsa-miR-769-3p | 0 | 16.82 | 0.0014 |
| hsa-miR-373* | 0 | 6.74 | 0.0017 |
| hsa-miR-518c* | 0 | 3.65 | 0.0018 |
| hsa-miR-617 | 0 | 4.90 | 0.0019 |
| hsa-miR-492 | 0 | 184.11 | 0.0019 |
| hsa-miR-557 | 0 | 6.24 | 0.0022 |
| hsa-miR-181b_v9.1 | 0 | 3.07 | 0.0023 |
| hsa-miR-489 | 0 | 5.47 | 0.0024 |
| hsa-miR-622 | 0 | 5.90 | 0.0027 |
| hsa-miR-525-5p | 0 | 4.95 | 0.0029 |
| hsa-miR-518f*_v9.1 | 0 | 5.56 | 0.0031 |
| hsa-miR-520d*_v9.1 | 0 | 4.94 | 0.0034 |
| hsa-miR-516b | 0 | 3.78 | 0.0039 |
| hsa-let-7e_v9.1 | 0 | 3.55 | 0.0040 |
| hsa-miR-454* | 0 | 9.99 | 0.0040 |
| hsa-miR-544_v9.1 | 0 | 3.92 | 0.0040 |
| hsa-miR-191* | 0 | 6.31 | 0.0042 |
| hsa-miR-195 | 0 | 7.91 | 0.0044 |
| hsa-miR-634 | 0 | 28.76 | 0.0045 |
| hsa-miR-765 | 0 | 3.60 | 0.0046 |
| hsa-miR-495_v9.1 | 0 | 6.29 | 0.0048 |
| hsa-miR-202_v9.1 | 0 | 4.63 | 0.0048 |
| hsa-miR-625_v9.1 | 0 | 4.03 | 0.0050 |
| hsa-miR-452_v9.1 | 0 | 5.73 | 0.0052 |
| hsa-miR-567 | 0 | 12.78 | 0.0052 |
| hsa-miR-526a_v9.1 | 0 | 5.85 | 0.0053 |
| hsa-miR-526b_v9.1 | 0 | 6.62 | 0.0057 |
| hsa-miR-378* | 0 | 6.56 | 0.0063 |
| hsa-miR-302c* | 0 | 6.99 | 0.0064 |
| hsa-miR-601 | 0 | 6.36 | 0.0064 |
| hsa-miR-155_v9.1 | 0 | 5.08 | 0.0065 |
| hsa-miR-149_v9.1 | 0 | 3.94 | 0.0066 |
| hsa-miR-370_v9.1 | 0 | 3.97 | 0.0068 |
| hsa-miR-583 | 0 | 9.29 | 0.0072 |
| hsa-miR-182_v9.1 | 0 | 5.81 | 0.0077 |
| hsa-miR-182* | 0 | 3.48 | 0.0077 |
| hsa-miR-636_v9.1 | 0 | 2.62 | 0.0081 |
| hsa-miR-593* | 0 | 7.49 | 0.0081 |
| hsa-miR-184 | 0 | 5.59 | 0.0082 |
| hsa-miR-200c_v9.1 | 0 | 4.72 | 0.0082 |
| hsa-miR-597 | 0 | 4.00 | 0.0087 |
| hsa-miR-188_v9.1 | 0 | 5.21 | 0.0090 |
| hsa-miR-498 | 0 | 5.50 | 0.0093 |
| hsa-miR-600 | 0 | 4.40 | 0.0094 |
| hsa-miR-423_v9.1 | 0 | 5.92 | 0.0100 |
| hsa-miR-24-1* | 0 | 6.69 | 0.0103 |
| hsa-miR-490-3p | 0 | 6.15 | 0.0105 |
| hsa-miR-524-5p | 0 | 5.33 | 0.0105 |
| hsa-miR-100 | 0 | 4.11 | 0.0106 |
| hsa-miR-302d | 0 | 3.81 | 0.0110 |
| hsa-miR-550* | 0 | 4.98 | 0.0113 |
| hsa-miR-421 | 0 | 6.07 | 0.0115 |
| hsa-miR-518d-3p | 0 | 12.68 | 0.0116 |
| hsa-miR-576_v9.1 | 0 | 3.87 | 0.0119 |
| hsa-miR-610 | 0 | 3.66 | 0.0122 |
| hsa-miR-210 | 0 | 2.80 | 0.0125 |
| hsa-miR-589* | 0 | 7.97 | 0.0126 |
| hsa-miR-363* | 0 | 11.46 | 0.0130 |
| hsa-miR-630 | 0 | 10.74 | 0.0135 |
| hsa-miR-562 | 0 | 3.97 | 0.0136 |
| hsa-miR-211 | 0 | 3.76 | 0.0142 |
| hsa-miR-92b_v9.1 | 0 | 7.30 | 0.0144 |
| hsa-miR-659 | 0 | 8.69 | 0.0148 |
| hsa-miR-337_v9.1 | 0 | 6.22 | 0.0148 |
| hsa-miR-618 | 0 | 4.23 | 0.0149 |
| hsa-miR-525*_v9.1 | 0 | 3.90 | 0.0149 |
| hsa-miR-598 | 0 | 3.45 | 0.0149 |
| hsa-miR-9* | 0 | 3.27 | 0.0153 |
| hsa-miR-518e_v9.1 | 0 | 3.69 | 0.0156 |
| hsa-miR-214_v9.1 | 0 | 8.80 | 0.0159 |
| hsa-miR-591 | 0 | 4.46 | 0.0162 |
| hsa-miR-216_v9.1 | 0 | 5.44 | 0.0162 |
| hsa-miR-663 | 0 | 16.23 | 0.0164 |
| hsa-miR-770-5p | 0 | 5.04 | 0.0170 |
| hsa-miR-508-3p | 0 | 9.72 | 0.0173 |
| hsa-miR-154* | 0 | 3.34 | 0.0174 |
| hsa-miR-596 | 0 | 3.52 | 0.0175 |
| hsa-miR-623 | 0 | 4.56 | 0.0175 |
| hsa-miR-30c | 0 | 6.92 | 0.0175 |
| hsa-miR-219-5p | 0 | 10.28 | 0.0179 |
| hsa-miR-602 | 0 | 6.36 | 0.0183 |
| hsa-miR-619 | 0 | 3.37 | 0.0184 |
| hsa-miR-510_v9.1 | 0 | 3.87 | 0.0185 |
| hsa-miR-612 | 0 | 3.04 | 0.0188 |
| hsa-miR-768-3p_v11.0 | 0 | 4.29 | 0.0195 |
| hsa-miR-206 | 0 | 3.25 | 0.0197 |
| hsa-miR-200a | 0 | 4.56 | 0.0200 |
| hsa-miR-646 | 0 | 4.33 | 0.0200 |
| hsa-miR-516a-3p | 0 | 15.88 | 0.0202 |
| hsa-miR-518b | 0 | 4.64 | 0.0203 |
| hsa-miR-371-3p | 0 | 8.54 | 0.0210 |
| hsa-miR-656 | 0 | 58.25 | 0.0211 |
| hsa-miR-422a_v9.1 | 0 | 4.70 | 0.0217 |
| hsa-miR-512-5p | 0 | 4.44 | 0.0221 |
| hsa-miR-548a-3p | 0 | 3.69 | 0.0222 |
| hsa-miR-411 | 0 | 4.30 | 0.0223 |
| hsa-miR-323-3p | 0 | 3.57 | 0.0223 |
| hsa-miR-521 | 0 | 3.57 | 0.0223 |
| hsa-miR-607 | 0 | 5.17 | 0.0234 |
| hsa-miR-153_v9.1 | 0 | 8.54 | 0.0236 |
| hsa-miR-563 | 0 | 3.50 | 0.0237 |
| hsa-miR-647 | 0 | 2.72 | 0.0237 |
| hsa-miR-653_v9.1 | 0 | 4.03 | 0.0240 |
| hsa-miR-212 | 0 | 5.82 | 0.0242 |
| hsa-miR-329 | 0 | 3.32 | 0.0251 |
| hsa-miR-501-5p | 0 | 4.23 | 0.0255 |
| hsa-miR-181d_v9.1 | 0 | 3.42 | 0.0269 |
| hsa-miR-542-5p_v9.1 | 0 | 7.35 | 0.0285 |
| hsa-miR-650 | 0 | 9.52 | 0.0297 |
| hsa-miR-452*_v9.1 | 0 | 6.99 | 0.0298 |
| hsa-miR-432 | 0 | 3.07 | 0.0301 |
| hsa-miR-217_v9.1 | 0 | 10.93 | 0.0301 |
| hsa-miR-611 | 0 | 10.18 | 0.0303 |
| hsa-miR-616* | 0 | 3.25 | 0.0304 |
| hsa-miR-345_v9.1 | 0 | 12.42 | 0.0305 |
| hsa-miR-548d-3p | 0 | 4.45 | 0.0315 |
| hsa-miR-380* | 0 | 6.71 | 0.0320 |
| hsa-miR-380 | 0 | 5.93 | 0.0320 |
| hsa-miR-518c_v9.1 | 0 | 2.83 | 0.0325 |
| hsa-miR-30a* | 0 | 2.28 | 0.0328 |
| hsa-miR-560_v9.1 | 0 | 11.43 | 0.0349 |
| hsa-miR-99a | 0 | 6.89 | 0.0353 |
| hsa-miR-124a_v9.1 | 0 | 4.07 | 0.0367 |
| hsa-miR-515-5p | 0 | 20.94 | 0.0375 |
| hsa-miR-296-5p | 0 | 19.61 | 0.0381 |
| hsa-miR-30b | 0 | 3.99 | 0.0395 |
| hsa-miR-335 | 0 | 28.17 | 0.0396 |
| hsa-miR-579_v9.1 | 0 | 9.38 | 0.0399 |
| hsa-miR-10a | 0 | 3.28 | 0.0402 |
| hsa-miR-520b | 0 | 2.05 | 0.0426 |
| hsa-miR-376a*_v9.1 | 0 | 4.75 | 0.0427 |
| hsa-miR-375 | 0 | 3.46 | 0.0440 |
| hsa-miR-587 | 0 | 5.46 | 0.0453 |
| hsa-miR-572 | 0 | 10.02 | 0.0464 |
| hsa-miR-126* | 0 | 6.24 | 0.0464 |
| hsa-miR-205 | 0 | 11.27 | 0.0469 |
| hsa-miR-549 | 0 | 10.58 | 0.0486 |
| hsa-miR-564 | 0 | 5.66 | 0.0492 |

TABLE 4
List of Potential Circulating miR Candidates from Serum
Microarray Data Analyzed Using Sigificance of
Analysis Microarrays (SAM)
Hur-normalized serum microarray data (36 EAC, 12
Normal) was analyzed by SAM. 144 miRs elevated in
EAC serum were selected based on their 0% q-value,
significant p-value, and fold change greater than
2. Top 23 candidate miRs are shown in the table.

| Gene ID | Fold Change EAC/N | P-Value |
|---|---|---|
| hsa-miR-519e* | 9.36 | 0.0004 |
| hsa-miR-520a-5p | 12.43 | 0.0009 |
| hsa-miR-181a* | 5.99 | 0.0014 |
| hsa-miR-769-3p | 16.82 | 0.0014 |
| hsa-miR-373* | 6.74 | 0.0017 |
| hsa-miR-191* | 6.31 | 0.0042 |
| hsa-miR-195 | 7.91 | 0.0044 |
| hsa-miR-634 | 28.76 | 0.0045 |
| hsa-miR-765 | 3.6 | 0.0046 |
| hsa-miR-421 | 6.07 | 0.0115 |
| hsa-miR-518d-3p | 12.68 | 0.0116 |
| hsa-miR-630 | 10.74 | 0.0135 |
| hsa-miR-92b_v.9.1 | 7.3 | 0.0144 |
| hsa-miR-659 | 8.69 | 0.0148 |
| hsa-miR-663 | 16.23 | 0.0164 |
| hsa-miR-770-5p | 5.04 | 0.0170 |
| hsa-miR-508-3p | 9.72 | 0.0173 |
| hsa-miR-219-5p | 10.28 | 0.0179 |
| hsa-miR-200a | 4.56 | 0.0200 |
| hsa-miR-646 | 1.33 | 0.0200 |
| hsa-miR-345 | 12.42 | 0.0305 |
| hsa-miR-548d-3p | 4.45 | 0.0315 |
| hsa-miR-380* | 6.71 | 0.0320 |

TABLE 5
List of Potential Circulating miR Candidates from Serum
Microarray Data Analyzed Using GeneSpring GX11.5 Software
The microarray data was normalized and analyzed by GeneSpring
GX 11.5 software. Total 7 miRs elevated in EAC serum were
selected and shown in the table. MiR candidates that commonly
appear in this table and Table 5 are underlined.

| Gene ID | Fold Change EAC/N | P-Value |
|---|---|---|
| hsa-miR-345 | 4.94 | 0.0152 |
| hsa-miR-193a | 5.53 | 0.0152 |
| hsa-miR-328 | 4.11 | 0.0230 |
| hsa-miR-663 | 3.43 | 0.0304 |
| hsa-miR-373* | 1.95 | 0.0304 |
| hsa-miR-629 | 4.49 | 0.0345 |
| hsa-miR-630 | 3.75 | 0.0409 |

TABLE 6
List of Potential Circulating miR Candidates from Serum
Microarray Data Analyzed Using GeneSpring GX11.5 Software
Microarrays were done on normal esophageal (HEEPiC), Barrett's
(CHTRT, GiHTRT, QHTRT), and EAC (OE33) cell lines.
The data was Hur-normalized and analyzed by SAM. Top 12 miRs
elevated in EAC serum are shown. Many candidates were also
highly expressed in. Barrett's versus normal. MiR-345 is
underlined to show its common appearance in Tables 5 and 6.

Fold Changes

| Gene ID | BE/N | EAC/N |
|---|---|---|
| hsa-miR-625 | 6.375 | 24.917 |
| hsa-miR-25 | 2.273 | 9.160 |
| hsa-miR-93 | 1.356 | 8.122 |
| hsa-miR-106b | 1.477 | 7.386 |
| hsa-miR-155 | 3.417 | 4.481 |
| hsa-miR-345 | 2.542 | 4.107 |
| hsa-msR-130b | 1.440 | 3.347 |
| hsa-miR-429 | 0.005 | 3.346 |
| hsa-miR-28 | 1.028 | 2.583 |
| hsa-miR-30a-5p | 5.618 | 2.533 |
| hsa-miR-200a | 0.005 | 2.215 |
| hsa-miR-765 | 1.867 | 2.050 |

### Example 2

### Generation of MicroRNA Biomarker Panel for EAC Using a Different Normalization Method

The same data was normalized using GeneSpring GX 11.5, which uses quantile normalization (Example 1 used Har-based normalization). This method generated 51 (23 upregulated, 28 downregulated) possible miR candidates. Again, the cell line data which was processed in the same way as the serum data was also examined. The cell line data SAM result was used to confirm and narrow miR candidates. Fourteen miR candidates were selected for further validation (hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301a, hsa-miR-15b) which commonly appeared or were deemed significant in separate analysis.

TABLE 7
List of 51 Candidate MicroRNA Biomarkers
for EAC Using Quantile Normalization

| Gene ID | Regulation |
|---|---|
| hsa-miR-630 | UP |
| hsa-miR-345 | UP |
| hsa-miR-663 | UP |
| hsa-miR-328 | UP |
| hsa-miR-769-3p | UP |
| hsa-miR-572 | UP |
| hsa-miR-622 | UP |
| hsa-miR-584 | UP |
| hsa-miR-625 | UP |
| hsa-miR-93 | UP |
| hsa-miR-155 | UP |
| hsa-miR-130b | UP |
| hsa-miR-429 | UP |
| hsa-miR-186 | UP |
| hsa-miR-590 | UP |
| hsa-miR-339 | UP |
| hsa-miR-151 | UP |
| hsa-miR-28 | UP |
| hsa-miR-30a-5p | UP |
| hsa-miR-200a | UP |
| hsa-miR-301a | UP |
| hsa-miR-15b | UP |
| hsa-miR-765 | UP |
| hsa-miR-223 | DOWN |
| hsa-miR-22 | DOWN |
| hsa-miR-575 | DOWN |
| hsa-miR-92 | DOWN |
| hsa-miR-21 | DOWN |
| hsa-miR-30d | DOWN |
| hsa-miR-19b | DOWN |
| hsa-miR-24 | DOWN |
| hsa-miR-25 | DOWN |
| hsa-miR-106b | DOWN |
| hsa-miR-494 | DOWN |
| hsa-miR-197 | DOWN |
| hsa-miR-185 | DOWN |
| hsa-miR-138 | DOWN |
| hsa-miR-130a | DOWN |
| hsa-miR-377 | DOWN |
| hsa-miR-34c | DOWN |
| hsa-miR-100 | DOWN |
| hsa-miR-125b | DOWN |
| hsa-miR-136 | DOWN |
| hsa-miR-125a | DOWN |
| hsa-miR-34b | DOWN |
| hsa-miR-503 | DOWN |
| hsa-miR-203 | DOWN |
| hsa-miR-193a | DOWN |
| hsa-miR-146a | DOWN |
| hsa-miR-574 | DOWN |
| hsa-miR-27a | DOWN |

### Example 3

### Validation of miR-345

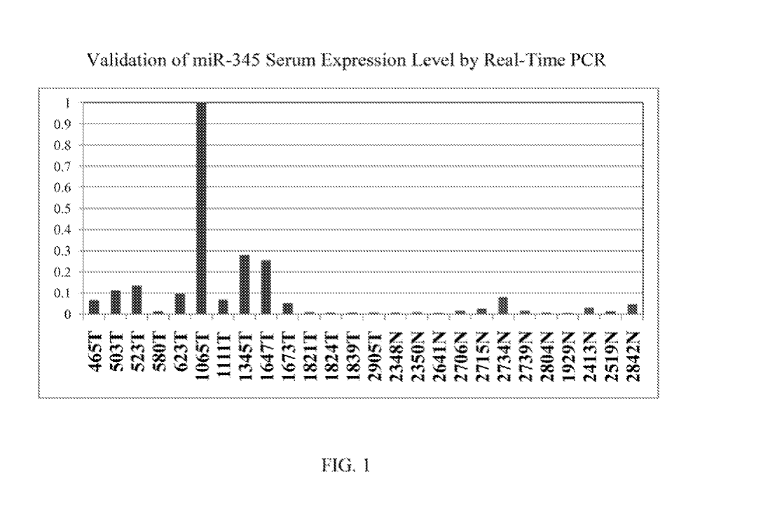

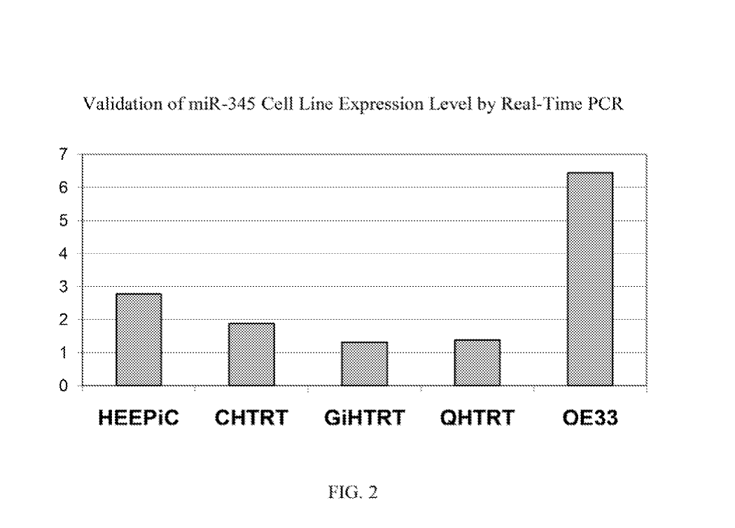

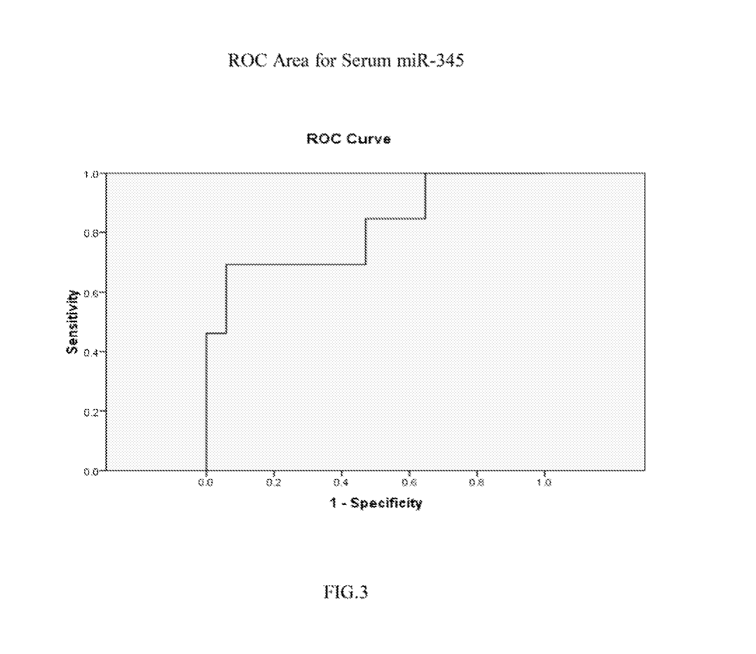

Real time PCR was performed on one of the selected miR candidates, hsa-miR-345, to validate its expression levels in sera and in cell lines. See FIG. 1 (serum expression level) and FIG. 2 (cell line expression level). MiR-345 reverse transcription was done by using TaqMan® MicroRNA Reverse Transcription Kit (Applied Biosystems, Inc. (Carlsbad, Calif.)) and its expression level was assessed in duplicate by real-time quantitative RT-PCR (qRT-PCR) using miR-345 specific probe provided by TaqMan® miR Assays (Applied Biosystems, Inc.). MiR-345 was amplified and normalized against miR-16 for serum and RNU6B for cell lines. The receiver operating characteristic (ROC) curve generated from serum levels of hsa-miR-345 yielded an area under the curve (AUC)=0.814, See FIG. 3. A panel consisting of several miRs after further validation is likely to disceriminate asymptomatic EAC patients from normal subjects, leading to earlier diagnosis and improved prognosis.

1. A method for diagnosing esophageal adenocarcinoma (EAC) in a patient comprising the steps of:
a. measuring the levels of at least one microRNA (miR) biomarker in a sample collected from the patient; and
b. comparing the levels of the at least one miR biomarker with corresponding levels in a control sample from a subject that does not have EAC,
wherein a significant difference in the levels of the at least one miR biomarker in the sample from the patient and the corresponding levels in the control sample is indicative that the patient has EAC.

2. The method of claim 1, wherein the at least one miR biomarker is selected from the group consisting of hsa-miR-345, hsa-miR-663, hsa-miR-373*, and hsa-miR-630.

3. The method of claim 1, wherein the at least one miR biomarker comprises hsa-miR-345, hsa-miR-663, hsa-miR-373*, and hsa-miR-630.

4. The method of claim 3, wherein the at least one miR biomarker further comprises one or more biomarkers selected from the group consisting of hsa-miR-193a, hsa-miR-328, hsa-miR-629.

5. The method of claim 1, wherein the at least one miR biomarker comprises hsa-miR-345, hsa-miR-663, hsa-miR-373*, hsa-miR-630, hsa-miR-193a, hsa-miR-328, and hsa-miR-629.

6. The method of claim 1, wherein the at least one miR biomarker comprises hsa-miR-625, hsa-miR-25, hsa-miR-93, hsa-miR-106b, hsa-miR-155, hsa-miR-345, hsa-miR-130b, hsa-miR-429, hsa-miR-28, hsa-miR-30a-5p, hsa-miR-200a, and hsa-miR-765.

7. The method of claim 1, wherein the at least one miR biomarker comprises hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b.

8. The method of claim 1, wherein the at least one miR biomarker comprises hsa-miR-519e, hsa-miR-520a-5p, hsa-miR-181a*, hsa-miR-769-3p, hsa-miR-373, hsa-miR-191*, hsa-miR-195, hsa-miR-634, hsa-miR-765, hsa-miR-421, hsa-miR-518d-3p, hsa-miR-630, hsa-miR-92b v9.1, hsa-miR-659, hsa-miR-663, hsa-miR-770-5p, hsa-miR-508-3p, hsa-miR-219-5p, hsa-miR-200a, hsa-miR-646, hsa-miR-345, hsa-miR-548d-3p, and hsa-miR-380*.

9. The method of claim 1, wherein the at least one miR biomarker comprises hsa-miR-630, hsa-miR-345, hsa-miR-663, hsa-miR-328, hsa-miR-769-3p, hsa-miR-572, hsa-miR-622, hsa-miR-584, hsa-miR-625, hsa-miR-93, hsa-miR-155, hsa-miR-130b, hsa-miR-429, hsa-miR-186, hsa-miR-590, hsa-miR-339, hsa-miR-151, hsa-miR-28, hsa-miR-30a-5p, hsa-miR-200a, hsa-miR-301a, hsa-miR-15b, and hsa-miR-765.

10. The method of claim 9, wherein the at least one miR biomarker further comprises one or more miR biomarkers selected from the group consisting of hsa-miR-223, hsa-miR-22, hsa-miR-575, hsa-miR-92, hsa-miR-21, hsa-miR-30d, hsa-miR-19b, hsa-miR-24, hsa-miR-25, hsa-miR-106b, hsa-miR-494, hsa-miR-197, hsa-miR-185, hsa-miR-138, hsa-miR-130a, hsa-miR-377, hsa-miR-34c, hsa-miR-100, hsa-miR-125b, hsa-miR-136, hsa-miR-125a, hsa-miR-34b, hsa-miR-503, hsa-miR-203, hsa-miR-193a, hsa-miR-146a, hsa-miR-574, and hsa-miR-27a.

11. The method of claim 1, wherein the sample is a serum sample.

12. The method of claim 1, wherein the sample is a plasma sample.

13. The method of claim 1, wherein the sample is a blood sample.

14. A method for diagnosing esophageal adenocarcinoma (EAC) in a patient comprising the steps of:
a. measuring the levels of a panel of microRNA (miR) biomarkers in a sample collected from the patient, wherein panel comprises hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b; and
b. comparing the levels of the panel of miR biomarkers with corresponding levels in a control sample from a subject that does not have EAC,
wherein a significant difference in the levels of the panel of miR biomarkers in the sample from the patient and the corresponding levels in the control sample is indicative that the patient has EAC.

15. The method of claim 14, wherein the sample is a blood, serum or plasma sample.

16. A method for determining the cancer status in a subject comprising the steps of:
a. measuring the levels of at least one biomarker in a sample collected from the subject; and
b. comparing the levels of the at least one biomarker with the corresponding levels in a control sample from a subject that does not have cancer and the corresponding levels in a control sample from a subject that has EAC,
wherein a correlation between the levels of the at least one biomarker in the sample from the subject and the corresponding levels in one of the control samples is determinative of the cancer status of the subject.

17. The method of claim 16, wherein the at least one biomarker measured comprises hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b.

18. The method of claim 16, wherein the sample is a blood, serum or plasma sample.

19. The method of claim 16, wherein the cancer is esophageal adenocarcinoma (EAC).

20. The method of claim 19, wherein the stage of EAC is determined.

21. The method of claim 19, wherein the progression of EAC is determined.

22. A kit for diagnosing EAC in a patient comprising miR hybridization or amplification reagents comprising one or more probe or amplification primers for hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b.

23. A kit for diagnosing EAC in a patient comprising miR hybridization or amplification reagents comprising one or more probe or amplification primers for a panel of miR biomarkers.

24. The kit of claim 23, wherein the panel of miR biomarkers comprises one or more miR biomarkers selected from the group consisting of hsa-miR-345, hsa-miR-663, hsa-miR-373*, and hsa-miR-630.

25. The kit of claim 23, wherein the panel of miR biomarkers comprises hsa-miR-345, hsa-miR-663, hsa-miR-373*, and hsa-miR-630.

26. The method of claim 25, wherein the panel of miR biomarkers further comprises one or more biomarkers selected from the group consisting of hsa-miR-193a, hsa-miR-328, hsa-miR-629.

27. The kit of claim 23, wherein the panel of miR biomarkers comprises hsa-miR-345, hsa-miR-663, hsa-miR-373*, hsa-miR-630, hsa-miR-193a, hsa-miR-328, and hsa-miR-629.

28. The kit of claim 23, wherein the panel of miR biomarkers comprises hsa-miR-625, hsa-miR-25, hsa-miR-93, hsa-miR-106b, hsa-miR-155, hsa-miR-345, hsa-miR-130b, hsa-miR-429, hsa-miR-28, hsa-miR-30a-5p, hsa-miR-200a, and hsa-miR-765.

29. The kit of claim 23, wherein the panel of miR biomarkers comprises hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301, and hsa-miR-15b.

30. The kit of claim 23, wherein the panel of miR biomarkers comprises hsa-miR-519e, hsa-miR-520a-5p, hsa-miR-181a*, hsa-miR-769-3p, hsa-miR-373, hsa-miR-191*, hsa-miR-195, hsa-miR-634, hsa-miR-765, hsa-miR-421, hsa-miR-518d-3p, hsa-miR-630, hsa-miR-92b v9.1, hsa-miR-659, hsa-miR-663, hsa-miR-770-5p, hsa-miR-508-3p, hsa-miR-219-5p, hsa-miR-200a, hsa-miR-646, hsa-miR-345, hsa-miR-548d-3p, and hsa-miR-380*.

31. The kit of claim 23, wherein the panel of miR biomarkers comprises hsa-miR-630, hsa-miR-345, hsa-miR-663, hsa-miR-328, hsa-miR-769-3p, hsa-miR-572, hsa-miR-622, hsa-miR-584, hsa-miR-625, hsa-miR-93, hsa-miR-155, hsa-miR-130b, hsa-miR-429, hsa-miR-186, hsa-miR-590, hsa-miR-339, hsa-miR-151, hsa-miR-28, hsa-miR-30a-5p, hsa-miR-200a, hsa-miR-301a, hsa-miR-15b, and hsa-miR-765.

32. The kit of claim 31, wherein the panel of miR biomarkers further comprises one or more miR biomarkers selected from the group consisting of hsa-miR-223, hsa-miR-22, hsa-miR-575, hsa-miR-92, hsa-miR-21, hsa-miR-30d, hsa-miR-19b, hsa-miR-24, hsa-miR-25, hsa-miR-106b, hsa-miR-494, hsa-miR-197, hsa-miR-185, hsa-miR-138, hsa-miR-130a, hsa-miR-377, hsa-miR-34c, hsa-miR-100, hsa-miR-125b, hsa-miR-136, hsa-miR-125a, hsa-miR-34b, hsa-miR-503, hsa-miR-203, hsa-miR-193a, hsa-miR-146a, hsa-miR-574, and hsa-miR-27a.
